(19) United States
(12) Patent Application Publication
(10) Pub. No.: US 2021/0398612 A1
(43) Pub. Date: Dec. 23, 2021

(54) DETECTING TUMOR MUTATION BURDEN WITH RNA SUBSTRATE

(71) Applicants: GeneCentric Therapeutics, Inc., Durham, NC (US); The University of North Carolina at Chapel Hill, Chapel Hill, NC (US)

(72) Inventors: Greg Mayhew, Durham, NC (US); Yoichiro Shibata, Durham, NC (US); Myla Lai-Goldman, Durham, NC (US); Charles Perou, Carrboro, NC (US); Joel Parker, Apex, NC (US)

(21) Appl. No.: 17/284,302

(22) PCT Filed: Oct. 9, 2019
(86) PCT No.: PCT/US2019/055322

§ 371 Date: Apr. 9, 2021

Related U.S. Application Data
(60) Provisional application No. 62/771,702, filed on Nov. 27, 2018.
(60) Provisional application No. 62/743,257, filed on Oct. 9, 2018.

(51) Int. Cl. G16B 20/20; G16B 30/10; G16B 50/10

(52) U.S. Cl. CPC G16B 20/20; G16B 50/10; G16B 30/10

(57) ABSTRACT

Methods and compositions are provided for determining TMB in a tumor sample using transcriptome profiling data. Also provided herein are methods and compositions for determining the response of an individual with a specific TMB to a therapy such as immunotherapy.

### CROSS-REFERENCE TO RELATED APPLICATIONS

This application claims the benefit of priority to U.S. Provisional Application No. 62/743,257 filed Oct. 9, 2018 and U.S. Provisional Application No. 62/771,702 filed Nov. 27, 2018, each of which is incorporated by reference herein in its entirety for all purposes.

### FIELD OF THE INVENTION

The present invention relates to methods for detecting the mutational load of somatic mutations from RNA isolated in a sample obtained from a subject suffering from or suspected of suffering from cancer. The present invention also relates to methods of determining prognosis of a subject suffering from or suspected of suffering from cancer based on the calculated tumor mutational burden rate.

### BACKGROUND OF THE INVENTION

Cancer cells accumulate mutations during cancer development and progression. These mutations may be the consequence of intrinsic malfunction of DNA repair, replication, or modification, or exposures to external mutagens. Certain mutations can confer growth advantages on cancer cells and can be positively selected in the microenvironment of the tissue in which the cancer arises. While the selection of advantageous mutations contributes to tumorigenesis, the likelihood of generating tumor neoantigens and subsequent immune recognition may also increase as mutations develop (Gubin and Schreiber. Science 350:158-9, 2015). Therefore, total mutation burden (TMB), can be used to guide patient treatment decisions, for example, to predict a durable response to a cancer immunotherapy. To date, elucidating TMB in various types of cancer has traditionally been done using whole exome sequencing (WES) or profiling a small fraction of the genome or exome such as described in, for example, WO2017151517. However, exome sequencing is not widely available, is expensive, time intensive, technically challenging, does not capture exons from mitochondria and may not capture desired exons as a result of exclusion during capture probe design. Moreover, while assessing TMB from genome or exome sequencing may aid in identifying candidate neoantigens, genome or exome sequencing data is not particularly useful for determining whether said candidate neoantigens are expressed in a tumor and ultimately available for antigen presentation to a patient's immune system. Further, genome or exome sequencing are not particularly useful for detecting RNAs that arise during alternative splicing or during RNA editing as described in Zhang et al., Nature Communication (2018) 9:3919.

Therefore, the need still exists for novel, cost-effective approaches, including transcriptomic profiling of the entire transcriptome or subsets thereof, to accurately measure mutational load in tumor samples.

### SUMMARY OF THE INVENTION

In one aspect, provided herein is a method of analyzing a tumor sample for a mutation load, comprising: detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germline variants; annotating the plurality of detected variants with annotation information from one or more population databases, wherein the population databases include information associated with variants in a population, wherein the annotation information includes missense status and germline alteration status associated with a given variant, thereby generating a plurality of annotated variants; filtering the plurality of annotated variants, wherein the filtering applies a rule set to the annotated variants to retain the detected variants that are non-synonymous somatic single nucleotide variants (SNVs), the rule set comprises: (i) removing SNVs corresponding to SNPs in a database of germline alterations; and (ii) removing SNVs not annotated as missense variants, wherein the filtering produces identified non-synonymous somatic SNVs; counting the identified non-synonymous somatic SNVs to give a tumor mutation value; determining a number of bases in the genomic regions targeted by the transcriptomic profile in the tumor sample genome; and calculating a number of non-synonymous somatic SNVs per megabase by dividing the tumor mutation value by the number of bases in the genomic regions targeted by the transcriptomic profile to produce the mutation load. In some cases, the population databases include one or more of a 1000 genomes database, Ensembl variation databases, COSMIC, Human Gene Mutation Database dbSNP, and an Exome Aggregation Consortium (ExAC) database. In some cases, the database of germline alterations in the dbSNP database. In some cases, the rule set further comprises removing the SNVs present in HLA and Ig genes and removing the SNVs with fewer than 25 total reads prior to (i). In some cases, the rule set further comprises removing SNPs having a reads ratio inconsistent with somatic mutation following step (ii), wherein the reads ratio equals reference allele reads/total reads. In some cases, the number of bases in the genomic regions targeted by the transcriptomic profile used to divide the tumor mutation value is multiplied by the percentage of bases with a desired sequencing depth. In some cases, the desired sequencing depth is 20×. In some cases, the genomic regions targeted by the transcriptomic profile are exons. In some cases, the detecting variants is configured by variant caller parameters, the variant caller parameters including a minimum allele frequency parameter, a strand bias parameter and a data quality stringency parameter. In some cases, prior to detecting variants, the method comprises aligning the nucleic acid sequence reads obtained from the transcriptomic profiling to a human reference genome; sorting and indexing; re-aligning to remove alignment errors and reference bias; and removing adjacent SNVs and indels. In some cases, the aligning the nucleic acid sequence reads obtained from the transcriptomic profiling to the human reference genome is performed with a spliced mapper.

In another aspect, provided herein is a system for analyzing a tumor sample genome for a mutation load, comprising a processor and a data store communicatively connected with the processor, the processor configured to perform the steps including: detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germ-line variants; annotating the plurality of detected variants with annotation information from one or more population databases, wherein the population databases include information associated with variants in a population, wherein the annotation information includes missense status and germline alteration status associated with a given variant, thereby generating a plurality of annotated variants; filtering the plurality of annotated variants, wherein the filtering applies a rule set to the annotated variants to retain the detected variants that are non-synonymous somatic single nucleotide variants (SNVs), the rule set comprises: (i) removing SNVs corresponding to SNPs in a database of germline alterations; and (ii) removing SNVs not annotated as missense variants, wherein the filtering produces identified non-synonymous somatic SNVs; counting the identified non-synonymous somatic SNVs to give a tumor mutation value; determining a number of bases in the genomic regions targeted by the transcriptomic profile in the tumor sample genome; and calculating a number of non-synonymous somatic SNVs per megabase by dividing the tumor mutation value by the number of bases in the genomic regions targeted by the transcriptomic profile to produce the mutation load. In some cases, the population databases include one or more of a 1000 genomes database, Ensembl variation databases, COSMIC, Human Gene Mutation Database dbSNP, and an Exome Aggregation Consortium (ExAC) database. In some cases, the database of germline alterations in the dbSNP database. In some cases, the rule set further comprises removing the SNVs present in HLA and Ig genes and removing the SNVs with fewer than 25 total reads prior to (i). In some cases, the rule set further comprises removing SNPs having a reads ratio inconsistent with somatic mutation following step (ii), wherein the reads ratio equals reference allele reads/total reads. In some cases, the number of bases in the genomic regions targeted by the transcriptomic profile used to divide the tumor mutation value is multiplied by the percentage of bases with a desired sequencing depth. In some cases, the desired sequencing depth is 20×. In some cases, the genomic regions targeted by the transcriptomic profile are exons. In some cases, the detecting variants is configured by variant caller parameters, the variant caller parameters including a minimum allele frequency parameter, a strand bias parameter and a data quality stringency parameter. In some cases, prior to detecting variants, the method comprises aligning the nucleic acid sequence reads obtained from the transcriptomic profiling to a human reference genome_{;} sorting and indexing; re-aligning to remove alignment errors and reference bias; and removing adjacent SNVs and indels. In some cases, the aligning the nucleic acid sequence reads obtained from the transcriptomic profiling to the human reference genome is performed with a spliced mapper.

In yet another aspect, provided herein is a non-transitory machine-readable storage medium comprising instructions which, when executed by a processor, cause the processor to perform a method analyzing a tumor sample genome for a mutation load, comprising: detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germ-line variants; annotating the plurality of detected variants with annotation information from one or more population databases, wherein the population databases include information associated with variants in a population, wherein the annotation information includes missense status and germline alteration status associated with a given variant, thereby generating a plurality of annotated variants; filtering the plurality of annotated variants, wherein the filtering applies a rule set to the annotated variants to retain the detected variants that are non-synonymous somatic single nucleotide variants (SNVs), the rule set comprises: (i) removing SNVs corresponding to SNPs in a database of germline alterations; and (ii) removing SNVs not annotated as missense variants, wherein the filtering produces identified non-synonymous somatic SNVs; counting the identified non-synonymous somatic SNVs to give a tumor mutation value; determining a number of bases in the genomic regions targeted by the transcriptomic profile in the tumor sample genome; and calculating a number of non-synonymous somatic SNVs per megabase by dividing the tumor mutation value by the number of bases in the genomic regions targeted by the transcriptomic profile to produce the mutation load.

In a still further aspect, provided herein is a method of identifying an individual having a cancer who may benefit from a cancer therapy, the method comprising determining a tumor mutational burden (TMB) rate using RNA sequencing data obtained from a tumor sample from the individual, wherein a TMB rate from the tumor sample that is at or above a reference TMB rate identifies the individual as one who may benefit from the cancer therapy.

In another aspect, provided herein is a method for selecting a cancer therapy for an individual having a cancer, the method comprising determining a TMB rate using RNA sequencing data from a tumor sample from the individual, wherein a TMB rate from the tumor sample that is at or above a reference TMB rate identifies the individual as one who may benefit from the cancer therapy.

In some cases, the TMB rate determined from the tumor sample is at or above the reference TMB rate, and the method further comprises administering to the individual an effective amount of the cancer therapy. In some cases, the TMB rate determined from the tumor sample is below the reference TMB rate.

In yet another aspect, provided herein is a method of treating an individual having a cancer, the method comprising: (a) determining a TMB rate from a tumor sample obtained from the individual, wherein the TMB rate from the tumor sample is at or above a reference TMB rate, and wherein the TMB rate is calculated from RNA sequencing data; and (b) administering a cancer therapy to the individual.

In some cases, the reference TMB rate is a pre-assigned TMB rate. In some cases, the reference TMB rate is between about 2 and about 5 mutations per megabase (mut/Mb). In some cases, the TMB rate determined using RNA sequencing data reflects a rate of non-synonymous somatic mutations. In some cases, the rate of non-synonymous somatic mutations represents a rate of candidate neoantigens. In some cases, the non-synonymous somatic mutations comprise mutations that have arisen due to RNA editing. In some cases, the tumor sample is from a patient suffering from or suspected of suffering from a type of cancer. The cancer can be a cervical kidney renal papillary cell carcinoma (KIRP); breast invasive carcinoma (BRCA); thyroid cancer (THCA); bladder carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head-neck squamous cell carcinoma (HNSC); uterine corpus endometrial carcinoma (EXEC), glioblastoma multiforme (GBM); esophageal carcinoma (ESCA); stomach adenocarcinorna (STAR); ovarian cancer (OV): rectum adenocarcinorna (READ) or lung squamous cell carcinoma (LUST). In some cases, the cancer is lung adenocarcinoma (LUAD); colon adenocarcinoma (COAD), breast invasive carcinoma (BRCA), uterine corpus endometrial carcinoma (UCEC), rectum adenocarcinoma (READ) or lung squamous cell carcinoma (LUSO. In some cases, the cancer therapy is selected from surgical intervention, radiotherapy, one or more chemotherapeutic agents, one or more PARP inhibitors, and one or more immunotherapeutic agents. In some cases, the one or more immunotherapeutic agents is an immune checkpoint modulator. In some cases, the immune checkpoint modulator interacts with cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof. In some cases, the immune checkpoint modulator is an antibody agent. In some cases, the antibody agent is or comprises a monoclonal antibody or antigen-binding fragment thereof. In some cases, the determining the TMB rate using RNA sequencing data comprises: detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germline variants; annotating the plurality of detected variants with annotation information from one or more population databases, wherein the population databases include information associated with variants in a population, wherein the annotation information includes missense status and germline alteration status associated with a given variant, thereby generating a plurality of annotated variants; filtering the plurality of annotated variants, wherein the filtering applies a rule set to the annotated variants to retain the detected variants that are non-synonymous somatic single nucleotide variants (SNVs), the rule set comprises: (i) removing SNVs corresponding to SNPs in a database of germline alterations; and (ii) removing SNVs not annotated as missense variants, wherein the filtering produces identified non-synonymous somatic SNVs; counting the identified non-synonymous somatic SNVs to give a tumor mutation value; determining a number of bases in the genomic regions targeted by the transcriptomic profile in the tumor sample genome; and calculating a number of non-synonymous somatic SNVs per megabase by dividing the tumor mutation value by the number of bases in the genomic regions targeted by the transcriptomic profile to produce the mutation load. In some cases, the population databases include one or more of a 1000 genomes database, Ensembl variation databases, COSMIC, Human Gene Mutation Database dbSNP, and an Exome Aggregation Consortium (ExAC) database. In some cases, the database of germline alterations in the dbSNP database. In some cases, the rule set further comprises removing the SNVs present in HLA and Ig genes and removing the SNVs with fewer than 25 total reads prior to (i). In some cases, the rule set further comprises removing SNPs having a reads ratio inconsistent with somatic mutation following step (ii), wherein the reads ratio equals reference allele reads/total reads. In some cases, the number of bases in the genomic regions targeted by the transcriptomic profile used to divide the tumor mutation value is multiplied by the percentage of bases with a desired sequencing depth. In some cases, the desired sequencing depth is 20×. In some cases, the genomic regions targeted by the transcriptomic profile are exons. In some cases, the detecting variants is configured by variant caller parameters, the variant caller parameters including a minimum allele frequency parameter, a strand bias parameter and a data quality stringency parameter. In some cases, prior to detecting variants, the method comprises aligning the nucleic acid sequence reads obtained from the transcriptomic profiling to a human reference genome; sorting and indexing; re-aligning to remove alignment errors and reference bias; and removing adjacent SNVs and indels. In some cases, the aligning the nucleic acid sequence reads obtained from the transcriptomic profiling to the human reference genome is performed with a spliced mapper. In some cases, the human reference genome is the GRCh38 human reference genome.

### BRIEF DESCRIPTION OF THE DRAWINGS

FIG. 1 illustrates a flow chart detailing the algorithm used to determine tumor mutational burden (TMB) value and TMB rate using TCGA RNA-seq fastq data.

FIG. 2 illustrates the process for normalizing SNV counts to only transcriptome targeted regions with high coverage (e.g. 20×, 50×, 100×) and example TMB calculations at specific coverages from one sample from a training data set.

FIG. 3 illustrates variations in the correlation of the RNA-seq TMB rate method (rTMB) with the gold standard TMB rate method at different coverage parameter values. The percent coverage represents the sequencing depth. The gold standard TMB rate method is based on assessing DNA sequence mutations as described in Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018, The immune landscape of cancer. Immunity, 48(4), pp. 812-830.

FIG. 4 illustrates variations in the correlation between the rTMB rate method with the gold standard TMB rate method at different reads ratio parameter values. The distance threshold represents the reads ratio, which is equal to the reference allele reads/total reads.

FIG. 5 illustrates correlations among rTMB estimates at several steps of the algorithm as well as with the gold standard TMB rate methods.

FIG. 6 illustrates the tumor mutation burden (TMB) rate calculated for 6 types of cancer using whole exome sequencing (WES) data obtained from the Cancer Genome Atlas (TCGA). This method of calculating TMB rate represents the gold standard method for determining TMB rate in a tumor sample. The legend details the number of samples (n) for each type of cancer. The types of cancer are bladder urothelial carcinoma (BLCA); lung adenocarcinoma (LUAD); colon adenocarcinoma (COAD); uterine corpus endometrial carcinoma (UCEC); rectum adenocarcinoma (READ); lung squamous cell carcinoma (LUSC); For LUAD, ⅔ of the sample (n=70) was used as a training set for the develop of an algorithm to calculate TMB rate from RNA-seq data as detailed in Example 1, while ⅓ (n=35) of the LUAD samples were used as a test set.

FIG. 7A-7B illustrates the correlation with the gold standard TMB rate for the RNA-seq TMB rate for the individual datasets for each cancer (i.e., FIG. 7A) and overall (i.e., FIG. 7B). The overall correlation analysis shown in FIG. 7B excludes the LUAD training set (n=70). Each of the plots in FIGS. 7A and 7B use log transformed values.

### DETAILED DESCRIPTION OF THE INVENTION

### Definitions

As used herein, the term “immune checkpoint modulator” refers to an agent that interacts directly or indirectly with an immune checkpoint. In some embodiments, an immune checkpoint modulator increases an immune effector response (e.g., cytotoxic T cell response), for example by stimulating a positive signal for T cell activation. In some embodiments, an immune checkpoint modulator increases an immune effector response (e.g., cytotoxic T cell response), for example by inhibiting a negative signal for T cell activation (e.g. disinhibition). In some embodiments, an immune checkpoint modulator interferes with a signal for T cell anergy. In some embodiments, an immune checkpoint modulator reduces, removes, or prevents immune tolerance to one or more antigens.

The term “modulator” as used herein can refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

The term “neoepitope” as used herein can refer to an epitope that emerges or develops in a subject after exposure to or occurrence of a particular event (e.g., development or progression of a particular disease, disorder or condition, e.g., infection, cancer, stage of cancer, etc.). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neoepitope is one that triggers an immune response that kills or otherwise destroys cells that express it (e.g., at a relevant level). In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope).

The term “sequence variant” (also called a variant) as used herein can correspond or refer to differences from a reference genome, which could be a constitutional genome of an organism or parental genomes. Examples of sequence variants can include a single nucleotide variant (SNV) and variants involving two or more nucleotides. Examples of SNVs include single nucleotide polymorphisms (SNPs) and point mutations. As examples, mutations can be “de novo mutations” (e.g., new mutations in the constitutional genome of a fetus) or “somatic mutations” (e.g., mutations in a tumor).

The term “somatic mutation” or “somatic alteration” can refer to a genetic alteration occurring in the somatic tissues (e.g., cells outside the germline). Examples of genetic alterations include, but are not limited to, point mutations (e.g., the exchange of a single nucleotide for another (e.g., silent mutations, missense mutations, and nonsense mutations)), insertions and deletions (e.g., the addition and/or removal of one or more nucleotides (e.g., indels)), amplifications, gene duplications, copy number alterations (CNAs), rearrangements, and splice variants. The presence of particular mutations can be associated with disease states (e.g., cancer).

The term “sequencing depth” as used herein can refer to the number of times a locus is covered by a sequence read aligned to the locus. The locus could be as small as a nucleotide, or as large as a chromosome arm, or as large as the entire genome. Sequencing depth can be expressed as 50 times, 100 times, etc., where “x” refers to the number of times a locus is covered with a sequence read. Sequencing depth can also be applied to multiple loci, or the whole genome, in which case x can refer to the mean number of times the loci or the whole genome, respectively, is sequenced. Ultra-deep sequencing can refer to at least 100 times in sequencing depth.

The term “sequencing breadth” can refer to what fraction of a particular reference genome (e.g., human) or part of the genome has been analyzed. The denominator of the fraction could be a repeat-masked genome, and thus 100% may correspond to all of the reference genome minus the masked parts. Any parts of a genome can be masked, and thus one can focus the analysis on any particular part of a reference genome. Broad sequencing can refer to at least 0.1% of the genome being analyzed, e.g., by identifying sequence reads that align to that part of a reference genome.

A “mutational load” of a sample is a measured value based on how many mutations are measured. The mutational load may be determined in various ways, such as a raw number of mutations, a density of mutations per number of bases, a percentage of loci of a genomic region that are identified as having mutations, the number of mutations observed in a particular amount (e.g. volume) of sample, and proportional or fold increase compared with the reference data or since the last assessment. A “mutational load assessment” refers to a measurement of the mutational load of a sample.

As used herein, the terms “individual,” “patient,” and “subject” are used interchangeably and can refer to any single animal, more preferably a mammal (including such non-human animals as, for example, dogs, cats, horses, rabbits, zoo animals, cows, pigs, sheep, and non-human primates) for which treatment is desired. In particular embodiments, the individual or patient herein is a human.

The term “tumor,” as used herein, can refer to all neoplastic cell growth and proliferation, whether malignant or benign, and all pre-cancerous and cancerous cells and tissues. The terms “cancer,” “cancerous,” and “tumor” are not mutually exclusive as referred to herein.

As used herein, the term “reference TMB score” or “reference rTMB score” can refers to a TMB or rTMB score against which another TMB score or rTMB is compared, e.g., to make a diagnostic, predictive, prognostic, and/or therapeutic determination. For example, the reference TMB or rTMB score may be a TMB or rTMB score in a reference sample, a reference population, and/or a pre-determined value.

The term “detection” can includes any means of detecting, including direct and indirect detection.

The term “level” can refers to the amount of a somatic mutation in a biological sample. The level can be measured by methods known to one skilled in the art. The level can be increased or decreased relative to or in comparison to a control such that the control is as an individual or individuals who are not suffering from the disease or disorder (e.g., cancer) or an internal control (e.g., a reference gene).

The terms “substantially” or “substantial” as used herein can mean substantially similar in function or capability or otherwise competitive to the products, items (e.g., type of cancer, nucleic acid complement), services or methods recited herein. Substantially similar products, items (e.g., type of cancer, nucleic acid complement), services or methods are at least 80%, 81%, 82%, 83%, 83%, 84%, 85%, 86%, 87%, 88%, 89%, 90%, 91%, 92%, 93%, 94%, 95%, 96%, 97%, 98%, 99% or 99.5% similar or the same as a product, item (e.g., type of cancer, nucleic acid complement), service or method recited herein.

### Overview

The present invention provides kits, compositions and methods for characterizing a sample obtained from an individual suffering from or suspected of suffering from a cancer. The sample can be any sample as provided herein. The cancer can be any cancer as provided herein. The characterization of the sample can entail isolating total RNA from the sample and subsequently analyzing the identity of the RNA present or expressed in the sample. The identity of the RNA present or expressed in the sample can entail obtaining sequencing data from the RNA isolated from the sample. The sequencing data can be obtained using any of the methods known in the art and/or provided herein for obtaining sequencing data from RNA. In one embodiment, characterization of the sample using the methods provided herein entails determining the tumor mutation burden (TMB), the subtype, the proliferation score, the level of immune activation or any combination thereof from RNA sequencing data obtained from the sample.

In one embodiment, characterization or analysis of a sample as provided herein obtained from an individual entails determining a tumor mutation burden (TMB) of the sample such that the TMB is determined from sequencing data obtained from RNA (e.g., RNA-Seq) isolated from the sample. TMB as determined or calculated from RNA sequencing data can be referred to as rTMB. The determination of rTMB can comprise isolating RNA from a sample obtained from an individual suffering from or suspected of suffering from a cancer, converting the isolated RNA to complementary DNA (cDNA), amplifying the cDNA using a primer extension reaction such as PCR; and sequencing said amplified cDNA. The isolation of RNA can be accomplished using any method known in the art and/or provided herein. Conversion of the RNA to cDNA and the subsequent amplification of said cDNA can be performed using any methods known in the art and/or provided herein. The sequencing of the amplified cDNA can be performed using a next generation sequencing (NGS) method known in the art and/or provided herein. The sequence reads obtained from NGS of the cDNA can correspond to or represent genomic regions targeted or covered by the RNA sequencing (e.g., transcriptomic profiling) of the sample. The rTMB can then be ascertained from the plurality of sequencing reads obtained from sequencing the amplified cDNA in a method that can generally comprise detecting variants in the plurality of sequence reads obtained from the sample (e.g., tumor sample as provided herein) to produce a plurality of detected variants, variant annotation, variant prioritization, and TMB score determination

Detection of the variants from the sequence reads when determining or calculating rTMB can entail mapping the reads to a reference genome. The reference genome can be a human reference genome. In one embodiment, the human reference genome is the GRCh38v22 (10.2014 release hg38) version of the GRCh38 human genome reference. Many different tools have been developed and can be used in the methods provided herein for mapping of the sequence reads obtained from the cDNA to the reference genome. Any methods known in that art that utilize Burrows—Wheeler Transformation (BWT) compression techniques, Smith—Waterman (SW) Dynamic programing algorithm or the combination of both in order to find the optimal alignment match can be used. Alignment tools useful for detecting variants in the rTMB methods provided herein can include Bowtie2 (see Wu TD, Nacu S, Fast and SNP-tolerant detection of complex variants and splicing in short reads Bioinformatics. 2010 Apr. 1; 26(7):873-81, which is incorporated herein by reference), BWA (see Li H, Handsaker B, Wysoker A, Fennell T, Ruan J, Homer N, Marth G, Abecasis G, Durbin R, 1000 Genome Project Data Processing Subgroup. The Sequence Alignment/Map format and SAMtools. Bioinformatics. 2009 Aug. 15; 25(16):2078-9, which is incorporated herein by reference), MOSAIK (see Zhou W, Chen T, Zhao H, Eterovic A K, Meric-Bernstam F, Mills G B, Chen K. Bias from removing read duplication in ultra-deep sequencing experiments. Bioinformatics. 2014 Apr. 15; 30(8):1073-1080, which is incorporated herein by reference) SHRiMP2 (see Homer N, Nelson SF. Improved variant discovery through local re-alignment of short-read next-generation sequencing data using SRMA. Genome Biol. 2010; 11(10):R99, which is incorporated herein by reference) genomic mapping and alignment program (GMAP; see Wu TD, Nacu S. Fast and SNP-tolerant detection of complex variants and splicing in short reads. Bioinformatics. 2010 Apr 1; 26(7):873-81, which is incorporated herein by reference) Novoalign V3 (see http://www.novocraft.com) or STAR (see Dobin A, Davis CA, Schlesinger F, Drenkow J, Zaleski C, Jha S, Batut P, Chaisson M, Gingeras TR. “STAR: ultrafast universal RNA-seq aligner”. Bioinformatics. 2013 Jan 1;29(1):15-21, which is incorporated herein by reference). In one embodiment, the alignment tool is STAR version 2.5.3a. In one embodiment, the detection of variants from the sequence reads entails mapping the sequence reads to a human reference genome (e.g., the GRCh38v22 (10.2014 release hg38) version of the GRCh38 human genome reference) using the STAR (e.g., version 2.5.3a) alignment tool.

Following alignment of the sequence reads, the detection of variants can entail post-alignment processing. After mapping reads to the reference genome, a multi-step post-alignment processing procedure can be performed on the detected variants in order to minimize the artifacts that may affect the quality of downstream variant calling. The post-alignment processing can entail sorting and indexing the sequence reads, realigning the sequence reads, removing adjacent SNPS/indels base quality score recalibration (BQSR), or any combination thereof. Sorting and indexing can be useful in removing read duplicates prior to variant calling and can be performed by tools such as Picard MarkDuplicates (see http://picard.sourceforge.net) and SAM-tools (see Li H, Handsaker B, Wysoker A, Fennell T, Ruan J, Homer N, Marth G, Abecasis G, Durbin R, 1000 Genome Project Data Processing Subgroup. The Sequence Alignment/Map format and SAMtools. Bioinformatics. 2009 Aug 15; 25(16):2078-9, which is incorporated herein by reference), or Sambamba (see A. Tarasov, A. J. Vilella, E. Cuppen, I. J. Nijman, and P. Prins. Sambamba: fast processing of NGS alignment formats. Bioinformatics, 2015, which is incorporated herein by reference). In one embodiment, the sorting and indexing is performed by the Sambamba tool, version v0.6.7_linux. Realignment of the sequence reads following sorting and indexing can be performed using SRMA (see Homer N, Nelson SF. Improved variant discovery through local re-alignment of short-read next-generation sequencing data using SRMA. Genome Biol. 2010; 11(10):R99, which is incorporated herein by reference), IndelRealigner (see McKenna A, Hanna M, Banks E, Sivachenko A, Cibulskis K, Kemytsky A, Garimella K, Altshuler D, Gabriel S, Daly M, DePristo M A. The Genome Analysis Toolkit: a MapReduce framework for analyzing next-generation DNA sequencing data. Genome Res. 2010 Sep; 20(9):1297-303, which is incorporated herein by reference), Bowtie2, BWA or STAR as described above. In some case, realignment can serve to identify indels and improve alignment quality thereof. Following realignment, the post-alignment processing can also entail removing adjacent SNPS/indels, which can be performed using SamTools (see Li, H.; Handsaker, B.; Wysoker, A.; Fennell, T.; Ruan, J.; Homer, N.; Marth, G.; Abecasis, G.; Durbin, R.; 1000 Genome Project Data Processing Subgroup (2009). “The Sequence Alignment/Map format and SAMtools”. Bioinformatics. 25 (16): 2078-2079, which is incorporated herein by reference). The version of SamTools can be version 1.6-1-gdd8cab5.

In the sequencing reads, each base is assigned with a Phred-scaled quality score generated by the sequencer, which represents the confidence of a base call. Base quality can be a critical factor for accurate variant detection in the downstream analysis. However, the machine-generated scores can often be inaccurate and systematically biased. In some cases, the rTMB method provided herein can entail BQSR, which can serve to improve the accuracy of confidence scores before variant calling. BQSR can take into account all reads per lane and analyze covariation among the raw quality score, machine cycle, and dinucleotide content of adjacent bases. A corrected Phred-scaled quality score can be reported following BQSR for each base in the read alignment. BQSR programs that can be used in the methods provided herein can be the BaseRecalibrator from the GATK suite, which McKenna A, Hanna M, Banks E, Sivachenko A, Cibulskis K, Kernytsky A, Garimella K, Altshuler D, Gabriel S, Daly M, DePristo M A. The Genome Analysis Toolkit: a MapReduce framework for analyzing next-generation DNA sequencing data. Genome Res. 2010 Sep.; 20(9):1297-303, which is incorporated herein by reference. Other well-established programs for use in the methods provided herein can include Recab from the NGSUtils suite (see Breese MR, Liu Y. NGSUtils: a software suite for analyzing and manipulating next-generation sequencing datasets. Bioinformatics. 2013 Feb. 15; 29(4):494-6, which is incorporated herein by reference) and the Bioconductor package ReQON (see Cabanski C R, Cavin K, Bizon C, Wilkerson M D, Parker J S, Wilhelmsen K C, Perou C M, Marron J S, Hayes D N. ReQON: a Bioconductor package for recalibrating quality scores from next-generation sequencing data. BMC Bioinformatics. 2012 Sep. 4; 130:221, which is incorporated herein by reference).

Following post-alignment processing, the detection of variants in the rTMB method can entail variant calling. Variant calling can be utilized in the TMB method in order to identify and distinguish somatic mutations in the sample from germline variants present in normal tissue. Variant calling can also be used to remove low quality and non-autosomal or non-X chromosomes. A number of tools useful in the rTMB methods provided herein have been developed to identify somatic mutations with paired tumor—normal samples. Exemplary tools for use in somatic variant calling in the rTMB methods provided herein include, but are not limited to deepSNV (see Gerstung M, Beisel C, Rechsteiner M, et al. Reliable detection of subclonal single-nucleotide variants in tumour cell populations. Nat Commun. 2012;3:811, which is incorporated herein by reference), Strelka (see Saunders C T, Wong W S W, Swamy S, Becq J, Murray L J, Cheetham R K. Strelka: accurate somatic small-variant calling from sequenced tumor—normal sample pairs. Bioinformatics. 2012; 28:1811-7, which is incorporated herein by reference), MutationSeq (see Ding J, Bashashati A, Roth A, et al. Feature-based classifiers for somatic mutation detection in tumour—normal paired sequencing data. Bioinformatics. 2012;28:167-75, which is incorporated herein by reference), MutTect, (see Cibulskis K, Lawrence MS, Carter SL, et al. Sensitive detection of somatic point mutations in impure and heterogeneous cancer samples. Nat Biotechnol. 2013; 31:213-9, which is incorporated herein by reference), QuadGT (http://www.iro.umontreal.ca/˜csuros/quadgt), Seurat (see Christoforides A, Carpten J D, Weiss G J, Demeure M J, Hoff D D V, Craig D W. Identification of somatic mutations in cancer through Bayesian-based analysis of sequenced genome pairs. BMC Genomics. 2013;14:302, which is incorporated herein by reference), Shimmer (see Hansen N F, Gartner J J, Mei L, Samuels Y, Mullikin J C. Shimmer: detection of genetic alterations in tumors using next-generation sequence data. Bioinformatics. 2013; 29:1498-503, which is incorporated herein by reference), and SolSNP (http://source-forge.net/projects/solsnp), jointSNVMix (see Roth A, Ding J, Morin R, et al. JointSNVMix: a probabilistic model for accurate detection of somatic mutations in normal/tumour paired next-generation sequencing data. Bioinformatics. 2012; 28:907-13, which is incorporated herein by reference), SomaticSniper (see Larson D E, Harris C C, Chen K, et al. SomaticSniper: identification of somatic point mutations in whole genome sequencing data. Bioinformatics. 2012; 28:311-7, which is incorporated herein by reference), VarScan2 (see Larson D E, Harris C C, Chen K, et al. SomaticSniper: identification of somatic point mutations in whole genome sequencing data. Bioinformatics. 2012; 28:311-7, which is incorporated herein by reference), MuSE, Mutect2 and Virmid (see Kim S, Jeong K, Bhutani K, et al. Virmid: accurate detection of somatic mutations with sample impurity inference. Genome Biol. 2013; 14:R90, which is incorporated herein by reference). In one embodiment, somatic variant calling is performed using Strelka2 (see Kim S. et al., Strelka2: fast and accurate calling of germline and somatic variants. Nature Methods, volume 15, pages591-594 (2018), which is incorporated herein by reference). The Strelka2 utilized can be version 2.9.0. In some cases, the detecting variants is configured by variant caller parameters, the variant caller parameters including a minimum allele frequency parameter, a strand bias parameter and a data quality stringency parameter.

Following variant detection and calling, the rTMB method provided herein can encompass variant annotation and prioritization. Different types of variants including SNVs, indels, CNVs, and large SVs can be detected from the sample by comparing the aligned reads to the reference genome, and can include both somatic variants and germline variants. As discussed herein, the post-alignment processing can encompass removal of adjacent SNPs and indels, and subsequent variant annotation and prioritization can yield the somatic TMB of the sample. In one embodiment, annotation of the somatic variants called can entail annotating the plurality of detected variants with annotation information from one or more population databases, wherein the population databases include information associated with variants in a population, wherein the annotation information includes missense status and germline alteration status associated with a given variant, thereby generating a plurality of annotated variants. The population databases can include one or more of a 1000 genomes database, Ensembl variation databases, ESP6500, COSMIC, Human Gene Mutation Database dbSNP, Complete Genomics personal genomes, NCI-60 human tumor cell line panel exome sequencing data, the LJB23 database, Combined Annotation Dependent Depletion (CADD) database, Phylop, Genomic Evolutionary Rate Profiling (GERP), PolyPhen and an Exome Aggregation Consortium (ExAC) database. In some cases, the database of germline alterations in the dbSNP database. The somatic variant annotation can be performed using any variant annotation tool known in the art. Exemplary annotation tools useful in the rTMB methods provided herein include, but are not limited to, ANNOVAR (see Wang K, Li M, Hakonarson H. ANNOVAR: functional annotation of genetic variants from high-throughput sequencing data. Nucleic Acids Res. 2010 September; 38(16):e164, which is incorporated herein by reference), SeattleSeq, VariantAnnotator from the GATK (see McKenna A, Hanna M, Banks E, Sivachenko A, Cibulskis K, Kernytsky A, Garimella K, Altshuler D, Gabriel S, Daly M, DePristo M A. The Genome Analysis Toolkit: a MapReduce framework for analyzing next-generation DNA sequencing data.Genome Res. 2010 Sep.; 20(9):1297-303, which is incorporated herein by reference) and SnpEff (see Cingolani P, Plans A, Wang le L, Coon M, Nguyen T, Wang L, Land S J, Lu X, Ruden D M. A program for annotating and predicting the effects of single nucleotide polymorphisms, SnpEff: SNPs in the genome of Drosophila melanogaster strain w1118; iso-2; iso-3. Fly (Austin). 2012 Apr.-Jun.; 6(2):80-92, which is incorporated herein by reference), or Variant Effect Predictor (see McLaren W, Gil L, Hunt S E, Riat H S, Ritchie G R, Thormann A, Flicek P, Cunningham F. The Ensembl Variant Effect Predictor. Genome Biology Jun. 6; 17(1):122. (2016), which is incorporated herein by reference.). In one embodiment, the annotation tool used in the rTMB method provided herien is VEP. The VEP used can be version ensembl-vep 91.3. The annotation can include SNP location, alleles, allele counts, missense status, dbSNP status and gene symbol.

Following annotation, the annotated variants can be prioritized by subjecting the annotated variants to a series of filtering steps. The filtering can comprise applying a rule set to the annotated variants to retain the detected variants that are non-synonymous somatic single nucleotide variants (SNVs). The rule set can comprise: (i) removing SNVs corresponding to SNPs in a database of germline alterations; and (ii) removing SNVs not annotated as missense variants, wherein the filtering produces identified non-synonymous somatic SNVs. Following variant prioritization, the rTMB value can be determined by counting the identified non-synonymous somatic SNVs. The rTMB rate or score can then be calculated by determining a number of bases in the genomic regions targeted by the transcriptomic profile in the tumor sample genome; and calculating a number of non-synonymous somatic SNVs per megabase by dividing the rTMB value by the number of bases in the genomic regions targeted by the transcriptomic profile to produce the mutation load. The total possible number of bases in the genomic regions targeted by the transcriptomic profile can be the number of bases covered by all exons with +/−10bp of flanking sequence. In one embodiment, the total possible number of bases in the genomic regions targeted by the transcriptomic profile is 135407705 bps. In some cases, the database of germline alterations in the dbSNP database. In some cases, the rule set further comprises removing the SNVs present in HLA and Ig genes and removing the SNVs with fewer than 25 total reads prior to (i). In some cases, the rule set further comprises removing SNPs having a reads ratio inconsistent with somatic mutation following step (ii), wherein the reads ratio equals reference allele reads/total reads. In some cases, the number of bases in the genomic regions targeted by the transcriptomic profile used to divide the tumor mutation value is multiplied by the percentage of bases with a desired sequencing depth. In some cases, the desired sequencing depth is 20×. In some cases, the genomic regions targeted by the transcriptomic profile are exons.

Prior to the detection of the variants during rTMB determination, quality control analysis of the raw sequence reads and preprocessing of the QC'd sequence reads can be performed. Quality control analysis of the raw sequence reads can comprise assessing the quality of raw NGS data. OC analysis can be performed using any one of the tools that include FastQC, FastQ Screen, FASTX-Toolkit, NGS QC Toolkit, PRINSEQ, QC-Chain and recently published QC3. Following the QC analysis, the sequencing reads can be subjected to pre-processing that can include base trimming, read filtering, or adaptor clipping. Several tools, such as Cutadapt and Trimmomatic, PRINSEQ and QC3 can be used to preprocess the sequence reads.

The rTMB method described herein can be implemented by a non-transitory machine-readable storage medium. The non-transitory machine-readable storage medium can be part of a data store that can be communicatively connected with a processor such that the non-transitory machine-readable storage medium comprises instructions which, when executed by a processor, perform the rTMB steps described herein for determining an rTMB score.

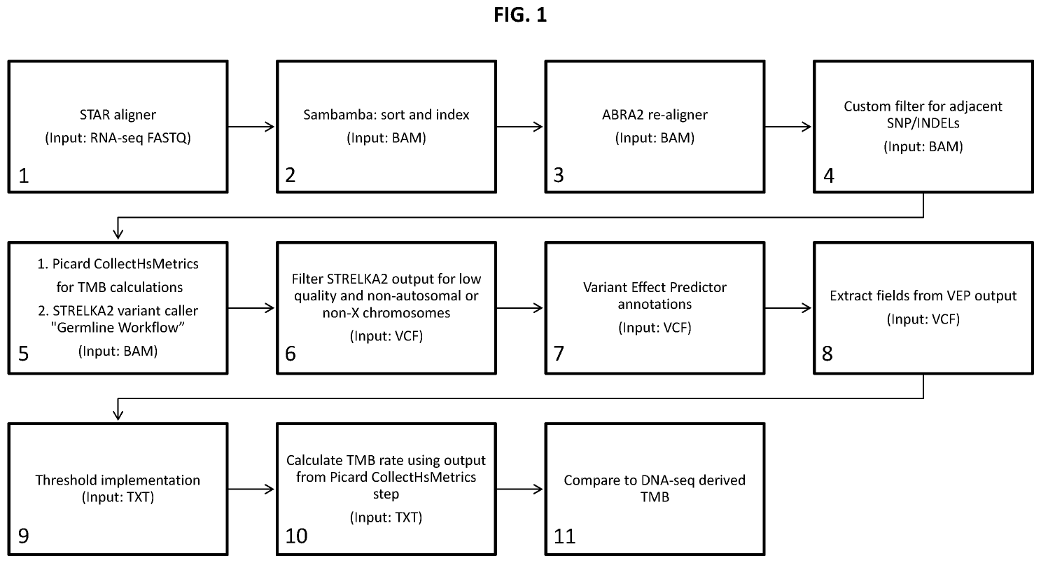

FIG. 1 depicts one exemplary embodiment of a method utilized to determine TMB value or score from RNA-sequencing data (e.g., transcriptomic profiling) obtained from a sample provided by an individual suffering from or suspected of suffering from a cancer. As shown in FIG. 1, the methods comprises aligning fastq converted RNA-seq data to a a human reference genome (i.e., the GRCh38v22 (10.2014 release hg38) version of the GRCh38 human genome reference) using STAR software' (version 2.5.3a; block 1 of FIG. 1), sorting and indexing reads using Sambamba software³ (version v0.6..7 linux; block 2 of FIG. 1), re-aligning reads using ABRA2⁴ (version abra2-2.14; block 3 of FIG. 1), removing adjacent SNP/Indels using SAMtools⁵ (version 1.6-1-gdd8cab5; block 4 of FIG. 1), determining a normalization factor for TMA rate calculations using Picard CollectHsMetrics and calling variants using STRELKA2⁶ (version strelka-2.9.0; block 5 of FIG. 1), removing low-confidence calls and non-canonical chromosomes (i.e. “chrUn”, “random”, “decoy”, “chrM”, “chrY”) using STRELKA² default filters (block 6 of FIG. 1), and annotating the remaining SNPs using Variant Effect Prediction' (VEP; version ensembl-vep 91.3 (cached, offline version); block 7 of FIG. 1) in order to facilitate further filtering of any remaining SNPs. The annotation included SNP location, alleles, allele counts, missense status, dbSNP status and gene symbol. The annotated SNPs can be subjected to a series of filtering steps. (i.e., blocks 8-10 of FIG. 1). The filtering and prioritization steps can include: (1) removing SNPs in HLA and IG genes (gene symbol starts with “HLA” or “IG”); (2) removing SNPs with fewer than 25 total reads; (3) removing SNPs in dbSNP (dbSNP version 150, which is used by VEP version 91); (4) removing SNPs not called “missense_variant” by VEP; (5) removing SNPs having a reads ratio not consistent with somatic mutation (i.e., SNPs with read ratios (reference allele reads/total reads) near 0, ¹/₂, or 1) and (6) converting the TMB value obtained from the preceding algorithm steps into a TMB rate or score. by normalizing the value to a transcriptome targeted region with high coverage (i.e., sequencing depth). Any of the alternative software tools provided herein can be used in place of those depicted in FIG. 1 in their respective step. The method depicted in FIG. 1 can be implemented by a non-transitory machine-readable storage medium. The non-transitory machine-readable storage medium can be part of a data store that can be communicatively connected with a processor such that the non-transitory machine-readable storage medium comprises instructions which, when executed by a processor, perform the steps outlined in FIG. 1 for determining an rTMB score.

In one embodiment, an rTMB score from a sample (e.g., tumor sample) from an individual is compared to a reference rTMB score. In some cases, the rTMB score from the tumor sample can be at or above a reference rTMB score and can identify the individual as one who may benefit from a treatment as described further herein. In some cases, the rTMB score from the tumor sample can be below a reference rTMB score and can identify the individual as one who may benefit from a treatment as described further herein.

In one embodiment, the reference rTMB score can be an rTMB score in a reference population of individuals having the cancer the individual from the which the sample used to calculate the tumor rTMB score suffers or is suspected of suffering from.

In another embodiment, the reference rTMB score is a pre-assigned rTMB score. In some instances, the reference rTMB score is between about 1 and about 100 mutations per Mb (mut/Mb), for example, about, 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mut/Mb. For example, in some instances, the reference rTMB score is between about 2 and about 30 mut/Mb (e.g., about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mut/Mb). In some instances, the reference rTMB score is between about 2 and about5 mut/Mb (e.g., about 2, about 3, about 4, or about 5 mut/Mb). In particular instances, the reference rTMB score may be 2 mut/Mb, or 5 mut/Mb.

In some cases, the tumor sample from the individual suffering from or suspected of suffering from a cancer has an rTMB score of greater than, or equal to, about 5 mut/Mb. For example, in some instances, the rTMB score from the tumor sample is between about 5 and about 100 mut/Mb (e.g., about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mut/Mb). In some instance, the tumor sample from the patient has an rTMB score of greater than, or equal to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 mut/Mb. For example, in some instances, the tumor sample from the patient has an rTMB score of greater than, or equal to, about 5 mut/Mb. In some instances, the rTMB score from the tumor sample is between about 5 and 100 mut/Mb. In some instances, the rTMB score from the tumor sample is between about 5 and 20 mut/Mb. In some instances, the tumor sample from the patient has an rTMB score of greater than, or equal to, about 10 mut/Mb. In some instances, the tumor sample from the patient has an rTMB score of greater than, or equal to, about 20 mut/Mb.

In some cases, the rTMB score or the reference rTMB score is represented as the number of somatic mutations counted per a defined number of sequenced bases. For example, in some instances, the defined number of sequenced bases is between about 100 kb to about 10 Mb. In some instances, the defined number of sequenced bases is about 1.1 Mb (e.g., about 1.125 Mb).

In one embodiment, MSI is assessed using a PCR-based approach such as the MSI Analysis System (Promega, Madison, Wis.), which is comprised of 5 pseudomonomorphic mononucleotide repeats (BAT-25, BAT-26, NR-21, NR-24, and MONO-27) to detect MSI and 2 pentanucleotide loci (PentaC and PendaD) to confirm identity between normal and tumor samples. The size in bases for each microsatellite locus can be determined, e.g., by gel electrophoresis, and a tumor may be designated MSI-H if two or more mononucleotide loci vary in length compared to the germline DNA. See, e.g., Le et al. NEJM 372:2509-2520, 2015.

In some embodiments, a somatic mutation results in a neoantigen or neoepitope. A neoepitope or neoantigen can contribute to increased binding affinity to MHC Class I molecules and/or recognition by cells of the immune system (i.e. T cells) as “non-self”. In one embodiment, the non-synonymous SNVs detected using the rTMB methods provided herein represent neoantigens or neoepitopes found in the sample obtained from the individual suffering from or suspected of suffering from a cancer. Further to this embodiment, the rTMB value and rTMB rate or score provides a direct measure of the neoantigen or neoepitope levels in the sample. In one embodiment, the levels of neoantigens or neoepitopes is useful for determining response of the individual to different cancer therapeutics. In some cases, a high rTMB score as compared to a reference rTMB score for an individual indicates an increased level of neoantigens and can identify the individual as one who may benefit from a treatment as described further herein. In some cases, a low rTMB score as compared to a reference rTMB score for an individual indicates a decreased level of neoantigens and can identify the individual as one who may benefit from a treatment as described further herein.

In one embodiment, characterization of a sample as provided herein obtained from an individual entails determining a subtype of the sample such that the subtype is determined from sequencing data obtained from RNA (e.g., RNA-Seq) isolated from the sample. The gene expression based cancer subtyping using RNA sequencing data can be determined using gene signatures known in the art for specific types of cancer. In one embodiment, the cancer is lung cancer and the gene signature is selected from the gene signatures found in WO2017/201165, WO2017/201164, US20170114416 or U.S. Pat. No. 8,822,153, each of which is herein incorporated by reference in their entirety. In one embodiment, the cancer is head and neck squamous cell carcinoma (HNSCC) and the gene signature is selected from the gene signatures found in PCT/US18/45522 or PCT/US18/48862, each of which is herein incorporated by reference in their entirety. In one embodiment, the cancer is breast cancer and the gene signature is the PAM50 subtyper found in Parker JS et al., (2009) Supervised risk predictor of breast cancer based on intrinsic subtypes. J Clin Oncol 27:1160-1167, which is herein incorporated by reference in its entirety.

In another embodiment, characterization of a sample as provided herein obtained from an individual entails determining an immune subtype of the sample such that the immune subtype is determined from sequencing data obtained from RNA (e.g., RNA-Seq) isolated from the sample. The gene expression based immune subtyping or immune cell activation using RNA sequencing data can be determined using immune expression signatures known in the art such as, for example, the gene signatures found in Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018, The immune landscape of cancer. Immunity, 48(4), pp. 812-830, which is herein incorporated by reference in its entirety. In one embodiment, immune cell activation is determined by monitoring the immune cell signatures of Bindea et al (Immunity 2013; 39(4); 782-795), the contents of which are herein incorporated by reference in its entirety. In one embodiment, the method further comprises measuring single gene immune biomarkers, such as, for example, CTLA4, PDCD1 and CD274 (PD-LI), PDCDLG2(PD-L2) and/or IFN gene signatures. In one embodiment, the level of immune cell activation is determined by measuring gene expression signatures of immunomarkers. The immunomarkers can be measured in the same and/or different sample used to determine the rTMB value and/or rate as described herein. The immunomarkers can be those found in WO2017/201165, and WO2017/201164, each of which is herein incorporated by reference in their entirety.

In yet another embodiment, characterization of a sample as provided herein obtained from an individual entails determining proliferation of the sample such that the proliferation is determined from sequencing data obtained from RNA (e.g., RNA-Seq) isolated from the sample. The gene expression based assessment of proliferation using RNA sequencing data can be determined using proliferation signatures known in the art for specific types of cancer such as, for example the PAM50 proliferation signature found in Nielsen T O et al., (2010) A comparison of PAM50 intrinsic subtyping with immunohistochemistry and clinical prognostic factors in tamoxifen-treated estrogen receptor positive breast cancer. Clin Cancer Res 16(21):5222-5232, which is herein incorporated by reference in its entirety.

In one embodiment, also provided herein are methods for utilizing RNA sequencing data generated nucleic acids isolated from a sample obtained from an individual suffering from or suspected of suffering from a cancer to determine the expression levels of of somatic mutations identified within said sample. The somatic mutations can be non-synonymous somatic mutations. The expression levels of the somatic mutations from the RNA sequencing data can be determined using any of the methods known in the art. For example, the expression levels of the somatic mutations from the RNA sequencing can be determined using the methods outlined in Ramskold D., Kayak E., Sandberg R. (2012) How to Analyze Gene Expression Using RNA-Sequencing Data. In: Wang J., Tan A., Tian T. (eds) Next Generation Microarray Bioinformatics. Methods in Molecular Biology (Methods and Protocols), vol 802, which is incorporated herein by reference.

### Sample Types

Further to any of the embodiments provided herein, a sample for use in the methods, compositions and kits provided herein can be a biological sample, such as a liquid biological sample or bodily fluid or a biological tissue. Examples of liquid biological samples or bodily fluids for use in the methods provided herein can include urine, blood, plasma, serum, saliva, ejaculate, stool, sputum, cerebrospinal fluid (CSF), tears, mucus, amniotic fluid or the like. Biological tissues are aggregates of cells, usually of a particular kind together with their intercellular substance that form one of the structural materials of a human or animal including connective, epithelium, muscle and nerve tissues. Examples of biological tissues also include organs, tumors, lymph nodes, arteries and individual cell(s). A biological tissue sample can be a biopsy. In one embodiment, the sample is a biopsy of a tumor, which can be referred to as a tumor sample. In one embodiment, the analyses described herein are performed on biopsies that are embedded in paraffin wax. Accordingly, the methods provided herein, including the RT-PCR methods, are sensitive, precise and have multi-analyte capability for use with paraffin embedded samples. See, for example, Cronin et al. (2004) Am. J Pathol. 164(1):35-42, herein incorporated by reference.

Formalin fixation and tissue embedding in paraffin wax is a universal approach for tissue processing prior to light microscopic evaluation. A major advantage afforded by formalin-fixed paraffin-embedded (FFPE) specimens is the preservation of cellular and architectural morphologic detail in tissue sections. (Fox et al. (1985) J Histochem Cytochem 33:845-853). The standard buffered formalin fixative in which biopsy specimens are processed is typically an aqueous solution containing 37% formaldehyde and 10-15% methyl alcohol. Formaldehyde is a highly reactive dipolar compound that results in the formation of protein-nucleic acid and protein-protein crosslinks in vitro (Clark et al. (1986) J Histochem Cytochem 34:1509-1512; McGhee and von Hippel (1975) Biochemistry 14:1281-1296, each incorporated by reference herein).

In one embodiment, the sample used herein is obtained from an individual, and comprises fresh-frozen paraffin embedded (FFPE) tissue.

The sample can be processed to render it competent for use in the methods provided herein that can entail fragmentation, ligation, denaturation, and/or amplification. Exemplary sample processing can include lysing cells of the sample to release nucleic acid, purifying the sample (e.g., to isolate nucleic acid from other sample components, which can inhibit enzymatic reactions), diluting/concentrating the sample, and/or combining the sample with reagents for further nucleic acid processing. In some examples, the sample can be combined with a restriction enzyme, reverse transcriptase, or any other enzyme of nucleic acid processing.

### Types of Cancer

Further to any of the embodiments provided herein, the cancer can include, but is not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. Examples of a cancer also include, but are not limited to, a lung cancer (e.g., a non-small cell lung cancer (NSCLC)), a kidney cancer (e.g., a kidney urothelial carcinoma or RCC), a bladder cancer (e.g., a bladder urothelial (transitional cell) carcinoma (e.g., locally advanced or metastatic urothelial cancer, including 1L or 2L+ locally advanced or metastatic urothelial carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycosis fungoides, a Merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor.

In some cases, the cancer is selected from a cervical kidney renal papillary cell carcinoma (KIRP); breast invasive carcinoma (BRCA); thyroid ancer (THCA); bladder carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head-neck squamous cell carcinoma (HNSC); uterine corpus endometrial carcinoma (UCEC); glioblastoma multiforme (GBM); esophageal carcinoma (LSCA); stomach adenocarcinoma (STAD); ovarian cancer (OV); rectum adenocarcinoma (READ) or lung squamous cell carcinoma (LUSC), an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, an endometrial cancer. In some cases, the cancer is lung adenocarcinoma (LUAD); colon adenocarcinoma (COAD), breast invasive carcinoma (BRCA), uterine corpus endometrial carcinoma (UCEC), rectum adenocarcinoma (READ) or lung squamous cell carcinoma (LUSC).

### Sequencing

Further to any of the embodiments provided herein, sequencing data from RNA is obtained by isolating RNA from a sample obtained from an individual, converting said RNA to complementary DNA (cDNA), and sequencing said cDNA.

Isolation of RNA from the sample can be performed using any of the methods known in the art. The RNA isolated from the sample can be total RNA or mRNA. RNA isolation can be performed using a purification kit, a buffer set and protease from commercial manufacturers, such as Qiagen (Valencia, Calif.), according to the manufacturer's instructions. In one embodiment, total RNA is isolated from the sample. Commercially available RNA isolation kits include Qiagen RNeasy mini-columns, MasterPureTM, Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155, incorporated by reference in its entirety for all purposes). In one embodiment, total RNA can be isolated from FFPE tissues as described by Bibikova et al. (2004) American Journal of Pathology 165:1799-1807, herein incorporated by reference. Likewise, the High Pure RNA Paraffin Kit (Roche) can be used. Paraffin is removed by xylene extraction followed by ethanol wash. RNA can be isolated from sectioned tissue blocks using the MasterPure Purification kit (Epicenter, Madison, Wis.); a DNase I treatment step is included. RNA can be extracted from frozen samples using Trizol reagent according to the supplier's instructions (Invitrogen Life Technologies, Carlsbad, Calif). Samples with measurable residual genomic DNA can be resubjected to DNasel treatment and assayed for DNA contamination. All purification, DNase treatment, and other steps can be performed according to the manufacturer's protocol. After total RNA isolation, samples can be stored at −80.degree. C. until use.

In a separate embodiment, mRNA is isolated from the sample. General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995).

Conversion of RNA to cDNA can be performed using any of the methods known in the art for such a conversion, such as using reverse transcriptase in an reverse transcription reaction. cDNA does not exist in vivo and therefore is a non-natural molecule. Besides cDNA not existing in vivo, cDNA is necessarily different than mRNA, as it includes deoxyribonucleic acid and not ribonucleic acid.

The cDNA can then be amplified, for example, by the polymerase chain reaction (PCR) or other amplification method known to those of ordinary skill in the art. For example, other amplification methods that may be employed include the ligase chain reaction (LCR) (Wu and Wallace, Genomics, 4:560 (1989), Landegren et al., Science, 241:1077 (1988), incorporated by reference in its entirety for all purposes, transcription amplification (Kwoh et al., Proc. Natl. Acad. Sci. USA, 86:1173 (1989), incorporated by reference in its entirety for all purposes), self-sustained sequence replication (Guatelli et al., Proc. Nat. Acad. Sci. USA, 87:1874 (1990), incorporated by reference in its entirety for all purposes), incorporated by reference in its entirety for all purposes, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety for all purposes. The product of this amplification reaction, i.e., amplified cDNA is also necessarily a non-natural product. First, as mentioned above, cDNA is a non-natural molecule. Second, in the case of PCR, the amplification process serves to create hundreds of millions of cDNA copies for every individual cDNA molecule of starting material.

The sequencing reaction can be performed using next generation sequencing (NGS). The NGS system used can be any NGS system known in the art. In one embodiment, the cDNA is amplified with primers that introduce an additional DNA sequence (e.g., adapter) onto the fragments (e.g., with the use of adapter-specific primers) that make the amplified cDNA amendable to an NGS sequencing platform.

The methods described herein can be useful for sequencing by the method commercialized by Illumina, as described U.S. Pat. Nos. 5,750,341; 6,306,597; and 5,969,119. Complementary DNA (cDNA) products can be prepared as described herein, and can then be denatured and can be randomly attached to the inside surface of flow-cell channels. Unlabeled nucleotides can be added to initiate solid-phase bridge amplification to produce dense clusters of double-stranded DNA. To initiate the first base sequencing cycle, four labeled reversible terminators, primers, and DNA polymerase can be added. After laser excitation, fluorescence from each cluster on the flow cell can be imaged. The identity of the first base for each cluster can then be recorded. Cycles of sequencing are performed to determine the fragment sequence one base at a time.

In some embodiments, the methods. described herein are useful for preparing cDNA for sequencing by the sequencing by ligation methods commercialized by Applied Biosystems SOLiD sequencing). In other embodiments, the methods are useful for preparing cDNA for sequencing by synthesis using the methods commercialized by 454/Roche Life Sciences, including but not limited to the methods and apparatus described in Margulies et al. Nature(2005) 437:376-380 (2005); and U.S. Pat. Nos. 7,244,559, 7,335,762; 7,211,390; 7,244,567; 7,264,929; and 7,323,305. In other embodiments, the methods are useful for preparing cDNA for sequencing by the methods commercialized by Helicos. BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US200801.03058. In other embodiments, the methods are useful for preparing cDNA for sequencing by the methods commercialized by Pacific Biosciences as described in U.S. Pat. Nos. 7,462,452; 7,476,504; 7,405,281; 7,170,050; 7,462,468 7,476,503; 7,315,019; 7,302,146; 7,313,308; and US Application Publication Nos. US20090029385: US20090068655; US2009002431; and US20080206764.

Another example of a sequencing technique that can be used in the methods described heroin is nanopore sequencing (see e.g. Soni G V and Meller A. (2007) Clin Chem 53: 1996-2001). A nanopore can be a small hole of the order of 1 nanometer in diameter. Immersion of a nanopore in a conducting fluid and application of a potential across it can result in a slight electrical current due to conduction of ions through the nanopore. The amount of current that flows can be sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Another example of a sequencing technique that can be used in the methods described herein is semiconductor sequencing provided by Ion Torrent (e.g., using the ion Personal Genome Machine (KM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) are released in the well, which can be detected by the ion sensor. The semiconductor chip can then he washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

Another example of a sequencing technique that can he used in the methods described herein is nanoball sequencing (as performed, e.g., by Complete Genomics; see e.g., Drmanac et al. (2010) Science 327: 78-81). cDNA can be isolated, fragmented, and size selected. For example, cDNA can be fragmented (e.g., by sonication) to a mean length of about 500 bp. Adapters (Ad!) can be attached to the ends of the fragments. For example, cDNA can he fragmented with MspI and size selected to a mean length of about 500 bp. Adapters (Ad1) can be attached to the ends of the fragments. The adapters can be used to hybridize to anchors for sequencing reactions. cDNA with adapters bound to each end can be PCR amplified. The adapter sequences can be modified so that complementary single strand ends bind to each other forming circular DNA. The cDNA can be methylated to protect it from cleavage by a type IIS restriction enzyme used in a subsequent step. An adapter (e.g., the right adapter) can have a restriction recognition site, and the restriction recognition site can remain non-methylated. The non-methylated restriction recognition site in the adapter can be recognized by a restriction enzyme Acul), and the cDNA can be cleaved by Acul 13 by to the right of the right adapter to form linear double stranded cDNA. A second round of right and left adapters (Ad2) can he ligated onto either end of the linear cDNA, and all cDNA with both adapters hound can be PCR amplified (e.g., by PCR). Ad2 sequences can be modified to allow them to bind each other and form circular DNA. The DNA can be methylated, but a restriction enzyme recognition site can remain non-methylated on the left Ad1 adapter. A restriction enzyme (e.g., Acul) can be applied, and the DNA can be cleaved 13 hp to the left of ⁻the Adl to form a linear DNA fragment. A third round of right and left adapter (Ad3) can he ligated to the right and left flank of the linear DNA, and the resulting fragment can be PCR amplified. The adapters can be modified so that they can bind to each other and form circular DNA. A type III restriction enzyme (e.g., EcoP15) can he added; EcoP15 can cleave the DNA 26 bp to the left of Ad3 and 26 bp to the right of Ad2. This cleavage can remove a large segment of DNA and linearize the DNA once again. A fourth round of right and left adapters (Ad4) can be ligated to the DNA, the DNA can be amplified (e.g., by PCR), and modified so that they bind each other and form the completed circular DNA template. Rolling circle replication (e.g., using Phi 29 DNA polymerase) can he used to amplify small fragments of DNA. The four adapter sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adapter sequences can be determined.

In some cases, the sequencing technique can comprise paired-end sequencing in which both the forward and reverse template strand can be sequenced. In some cases, the sequencing technique can comprise mate pair library sequencing. In mate pair library sequencing, DNA can be fragments, and 2-5 kb fragments can be end-repaired (e.g., with biotin labeled dNTPs). The DNA fragments can be circularized. and non-circularized DNA can be removed by digestion. Circular DNA can be fragmented and purified using the biotin labels). Purified fragments can be end-repaired and ligated to sequencing adapters.

In some cases, a sequence read is about, more than about, less than about, or at least about 10, 11, 12, 13, 14, 15, 16. 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60. 61. 62, 63, 64, 65, 66, 67, 68, 69, 70, 71, 72, 73 74, 75, 76, 77, 78, 79, 80, 81, 82, 83, 84, 85, 86, 87, 88, 89, 90, 91, 92, 93, 94, 9.5, 96, 97, 98, 99, 100, 101, 102, 103, 104, 105, 106, 107, 108, 109, 110, 111, 112, 113, 114, 115, 116, 117 118, 119, 120, 121, 122, 123. 124. 125, 126, 127, 128, 129, 130, 131, 132. 133, 134, 135, 136, 137, 138, 139, 140, 141, 142, 143, 144, 145, 146, 147, 148, 149, 150, 151, 152, 153, 154. 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216. 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262. 263, 264, 265, 266, 267, 268, 269, 270, 271, 272, 273, 274, 275, 276, 277, 278, 279, 280. 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316 317, 318, 319, 320, 321, 322, 323, 324, 325, 326, 327, 328, 329, 330, 333 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364 365, 366, 367, 368, 369, 370, 371, 372, 373, 374, 375, 376, 377, 378, 379, 380, 381, 382. 383, 384, 385, 386. 387, 388, 389, 390, 391, 392, 393, 394, 395, 396. 397, 398, 399, 400, 401, 402, 403, 404, 405, 406, 407, 408, 409, 410, 411, 412, 413, 414. 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575. 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, or 3000 bases. In some cases, a sequence read is about 10 to about 50 bases, about 10 to about 100 bases, about 10 to about 200 bases. about 10 to about 300 bases, about 10 to about 400 bases, about 10 to about 500 bases, about 10 to about 600 bases, about 10 to about 700 bases, about 10 to about 800 bases, about 10 to about 900 bases, about 10 to about 1000 bases, about 10 to about 1500 bases, about 10 to about 2000 bases, about 50 to about 100 bases, about 50 to about 150 bases, about 50 to about 200 bases, about 50 to about 500 bases, about 50 to about 1000 bases, about 100 to about 200 bases, about 100 to about 300 bases, about 100 to about 400 bases, about 100 to about 500 bases, about 100 to about 600 bases, about 100 to about 700 bases. about 100 to about 800 bases, about 100 to about 900 bases, or about 100 to about 1000 bases.

The number of sequence reads from a sample can be about, more than about, less than about, or at least about 100, 1000, 5,000, 1.0,000, 20,000, 30,000, 40,000, 50,000, 60,000, 70,000, 80,000, 90,000, 100,000, 2.00,000, 300,000, 400,000, 500,000, 600.000, 700,000, 800,000, 900,000, 1,000,000, 2,000,000, 3,000.000, 4,000,000, 5,000,000, 6,000,000, 7,000,000, 8,000,000, 9,000,000, or 10,000,000.

The depth of sequencing of a sample can be about, more than about, less than about, or at least about 1×, 2×, 3×, 4×, 5×, 6×, 7×, 8×, 9×, 10×, 11×, 12×, 13×, 14×, 15×, 16×, 17×, 1.8×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 300×, 400×, 500×, 600×, 700×, 800×, 900×, 1000×, 1500×, 2000×, 2500×, 1000×, 1500×, 4000×, 4500×, 5000×, 6000×, 6500×, 7000×, 7500×, 8000×, 8500×, 9000×, 9500×, 10000×, 15,000×, 20,000×, 25,000×, 30,000×, or 35,000N. The depth of sequencing of a sample can about 1× to about 5×, about 1× to about 10×, about 1× to about 20×, about 5× to about 10×, about 5× to about 20×, about 5×, to about 30×, about 10× to about 20×, about 10× to about 25×, about 100× to about 30×, about 10× to about 40×, about 30× to about 100×, about 100× to about 200×, about 100× to about 500×, about 500× to about 1000×, about 1000×, to about 2000×, about 1000× to about 5000×, or about 5000× to about 10,000×. Depth of sequencing can be the number of times a sequence (e.g., a transcript) is sequenced. In some cases, the Lander/Waterman equation is used for computing coverage. The general equation can be: C=LN/G, where C=coverage; G=haploid genome length, L=read length; and N=number of reads. As provided herein, the sequencing depth can be utilized to determine TMB. In one embodinyent, a sequencing depth of 20× is utilized by the methods provided herein to calculate TMB value and/or rate. In order to determine the optimal coverage or sequencing depth necessary for the TMB rate calculation, the sequencing data can be analyzed with the Picard CollectHsMetrics tool in order to get coverage output values. The use of the Picard CollectHsMetrics tool can be incorporated into the method for determining rTMB as provided herein.

Clinical/Therapeutic Uses

In one embodiment, the method as provided herein for characterizing a sample using RNA sequencing data obtained from a sample from a patient suffering or suspected of suffering from cancer is used to determine whether or not said patient is a candidate for treatment with a specific type or types of cancer therapy. The sample can be any type of sample obtained from the patient as provided herein. The cancer can be any type of cancer known in the art and/or provided herein. The characterization of the sample using the methods provided herein can entail determining the tumor mutation burden (TMB), the subtype, the proliferation score, the level of immune activation or any combination thereof from RNA sequencing data obtained from the sample. In one embodiment, the characterization is calculating a TMB value and/or rate from RNA (e.g., via transcriptome profiling or RNA sequencing)) as provided herein. The RNA based TMB value and/or rate (i.e., rTMB value and/or rTMB rate) for a sample obtained from a patient can be compared to a reference TMB rate and/or value. The reference TMB rate can be a pre-assigned TMB rate. In one embodiment, the reference TMB rate can be between about 2 and about 5 mutations per megabase (mut/Mb).

An rTMB value and/or rate from the sample obtained from the patient that is at or above a reference TMB value and/or rate identifies said patient as one who may benefit from a specific type or types of therapy. For example, an rTMB value and/or rate from the sample obtained from the patient that is at or above a reference TMB value and/or rate identifies said patient as one who may benefit from an immunotherapeutic agent (e.g., anti-PD-1 or anti-PD-L1 antibodies). Conversely, an rTMB value and/or rate from the sample obtained from the patient that is at or below a reference TMB value and/or rate identifies said patient as one who may not benefit from a specific type or types of therapy. For example, an rTMB value and/or rate from the sample obtained from the patient that is below a reference TMB value and/or rate identifies said patient as one who may not benefit from an immunotherapeutic agent (e.g., anti-PD-1 or anti-PD-L1 antibodies).

The determination of whether or not said patient is a candidate for treatment with a specific type or types of cancer therapy can be based on the calculated TMB value and/or rate from RNA alone or in combination with other methods known in the art for characterizing a sample obtained from a patient suffering from or suspected of suffering from cancer. The other methods for characterizing said sample can be histologically based methods, gene expression based methods or a combination thereof. The histologically based methods can include histological cancer subtyping by one or more trained pathologists as well as the histological based methods of assessing proliferation such as, for example, determining the mitotic activity index. The gene expression based methods can include subtyping, assessment of MSI, assessment of proliferation, assessment of cell of origin, immune subtyping or any combination thereof. The gene expression based methods can be assessed from DNA, RNA or a combination thereof. In one embodiment, the characterization of the sample obtained from the patient suffering from or suspected of suffering from cancer is performed on RNA obtained or isolated from the sample.

The gene expression based cancer subtyping can be determined using gene signatures known in the art for specific types of cancer. In one embodiment, the cancer is lung cancer and the gene signature is selected from the gene signatures found in WO2017/201165, WO2017/201164, US20170114416 or U.S. Pat. No. 8,822,153, each of which is herein incorporated by reference in their entirety. In one embodiment, the cancer is head and neck squamous cell carcinoma (HNSCC) and the gene signature is selected from the gene signatures found in PCT/US18/45522 or PCT/US18/48862, each of which is herein incorporated by reference in their entirety. In one embodiment, the cancer is breast cancer and the gene signature is the PAM50 subtyper found in Parker J S et al., (2009) Supervised risk predictor of breast cancer based on intrinsic subtypes. J Clin Oncol 27:1160-1167, which is herein incorporated by reference in its entirety.

The gene expression based immune subtyping or immune cell activation can be determined using immune expression signatures known in the art such as, for example, the gene signatures found in Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018. The immune landscape of cancer. Immunity, 48(4), pp. 812-830, which is herein incorporated by reference in its entirety. In one embodiment, immune cell activation is determined by monitoring the immune cell signatures of Bindea et al (Immunity 2013; 39(4); 782-795), the contents of which are herein incorporated by reference in its entirety. In one embodiment, the method further comprises measuring single gene immune biomarkers, such as, for example, CTLA4, PDCD1 and CD274 (PD-LI), PDCDLG2(PD-L2) and/or IFN gene signatures. In one embodiment, the level of immune cell activation is determined by measuring gene expression signatures of immunomarkers. The immunomarkers can be measured in the same and/or different sample used to determine the rTMB value and/or rate as described herein. The immunomarkers can be those found in WO2017/201165, and WO2017/201164, each of which is herein incorporated by reference in their entirety.

The gene expression based assessment of proliferation can be determined using proliferation signatures known in the art for specific types of cancer such as, for example the PAM50 proliferation signature found in Nielsen T O et al., (2010) A comparison of PAM50 intrinsic subtyping with immunohistochemistry and clinical prognostic factors in tamoxifen-treated estrogen receptor positive breast cancer. Clin Cancer Res 16(21):5222-5232, which is herein incorporated by reference in its entirety.

In one embodiment, upon determining a patient's rTMB value and/or rate alone or in combination with other characterization methods as described herein (e.g., cancer subtype, MSI, immune subtype and/or proliferation status), the patient is selected for a specific therapy, for example, radiotherapy (radiation therapy), surgical intervention, target therapy, chemotherapy or drug therapy with an angiogenesis inhibitor or immunotherapy or combinations thereof. In some embodiments, the specific therapy can be any treatment or therapeutic method that can be used for a cancer patient. In one embodiment, upon determining a patient's rTMB value and/or rate, the patient is administered a suitable therapeutic agent, for example chemotherapeutic agent(s) or an angiogenesis inhibitor or immunotherapeutic agent(s). In one embodiment, the therapy is immunotherapy, and the immunotherapeutic agent is a checkpoint inhibitor, monoclonal antibody, biological response modifier, therapeutic vaccine or cellular immunotherapy. In some embodiments, the determination of a suitable treatment can identify treatment responders. In some embodiments, the determination of a suitable treatment can identify treatment non-responders. In some embodiments, upon determining a patient's rTMB value and/or rate, the patient can be selected for any combination of suitable therapies. For example, chemotherapy or drug therapy with a radiotherapy, a surgical intervention with an immunotherapy or a chemotherapeutic agent with a radiotherapy. In some embodiments, immunotherapy, or immunotherapeutic agent can be a checkpoint inhibitor, monoclonal antibody, biological response modifier, therapeutic vaccine or cellular immunotherapy.

The methods of present invention are also useful for evaluating clinical response to therapy, as well as for endpoints in clinical trials for efficacy of new therapies.

In one embodiment, the methods of the invention also find use in predicting response to different lines of therapies based on the rTMB value and/or rate alone or in combination with other characterization methods as described herein (e.g., cancer subtype, immune subtype and/or proliferation status). For example, chemotherapeutic response can be improved by more accurately assigning rTMB value and/or rate. Likewise, treatment regimens can be formulated based on the rTMB value and/or rate alone or in combination with other characterization methods as described herein (e.g., cancer subtype, immune subtype and/or proliferation status).

### Angiogenesis Inhibitors

In one embodiment, upon determining a patient's rTMB value and/or rate alone or in combination with other characterization methods as described herein (e.g., cancer subtype, immune subtype and/or proliferation status), the patient is selected for drug therapy with an angiogenesis inhibitor.

In one embodiment, the angiogenesis inhibitor is a vascular endothelial growth factor (VEGF) inhibitor, a VEGF receptor inhibitor, a platelet derived growth factor (PDGF) inhibitor or a PDGF receptor inhibitor.

Each biomarker panel can include one, two, three, four, five, six, seven, eight or more biomarkers usable by a classifier (also referred to as a “classifier biomarker”) to assess whether a HNSCC patient is likely to respond to angiogenesis inhibitor therapy; to select a HNSCC patient for angiogenesis inhibitor therapy; to determine a “hypoxia score” and/or to subtype a HNSCC sample as basal, mesenchymal, atypical, or classical molecular subtype. As used herein, the term “classifier” can refer to any algorithm for statistical classification, and can be implemented in hardware, in software, or a combination thereof. The classifier can be capable of 2-level, 3-level, 4-level, or higher, classification, and can depend on the nature of the entity being classified. One or more classifiers can be employed to achieve the aspects disclosed herein.

In general, methods of determining whether a patient is likely to respond to angiogenesis inhibitor therapy, or methods of selecting a patient for angiogenesis inhibitor therapy are provided herein. In one embodiment, the method comprises determining an rTMB value and/or rate alone or in combination with other characterization methods as described herein (e.g., cancer subtype, immune subtype and/or proliferation status) and probing a sample from the patient for the levels of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 (see Table 1) at the nucleic acid level. In a further embodiment, the probing step comprises mixing the sample with five or more oligonucleotides that are substantially complementary to portions of nucleic acid molecules of the at least five biomarkers under conditions suitable for hybridization of the five or more oligonucleotides to their complements or substantial complements, detecting whether hybridization occurs between the five or more oligonucleotides to their complements or substantial complements; and obtaining hybridization values of the sample based on the detecting steps. The hybridization values of the sample are then compared to reference hybridization value(s) from at least one sample training set, wherein the at least one sample training set comprises (i) hybridization value(s) of the at least five biomarkers from a sample that overexpresses the at least five biomarkers, or overexpresses a subset of the at least five biomarkers, (ii) hybridization values of the at least five biomarkers from a reference basal, mesenchymal, atypical, or classical sample, or (iii) hybridization values of the at least five biomarkers from a HNSCC free head and neck sample. A determination of whether the patient is likely to respond to angiogenesis inhibitor therapy, or a selection of the patient for angiogenesis inhibitor is then made based upon (i) the patient's rTMB value and/or rate alone or in combination with other characterization methods as described herein (e.g., cancer subtype, immune subtype and/or proliferation status) and (ii) the results of comparison.

TABLE 1
Biomarkers for hypoxia profile

| Name | Abbreviation | GenBank Accession No. |
|---|---|---|
| RRAGD | Ras-related GTP binding D | BC003088 |
| FABP5 | fatty acid binding protein 5 | M94856 |
| UCHL1 | ubiquitin carboxyl-terminal | NM_004181 |
| | esterase L1 | |
| GAL | Galanin | BC030241 |
| PLOD | procollagen-lysine, | M98252 |
| | 2-oxoglutarate 5-dioxygenase | |
| | lysine hydroxylase | |
| DDIT4 | DNA-damage-inducible | NM_019058 |
| | transcript 4 | |
| VEGF | vascular endothelial | M32977 |
| | growth factor | |
| ADM | Adrenomedullin | NM_001124 |
| ANGPTL4 | angiopoietin-like 4 | AF202636 |
| NDRG1 | N-myc downstream regulated | NM_006096 |
| | gene 1 | |
| NP | nucleoside phosphorylase | NM 000270 |
| SLC16A3 | solute carrier family 16 | NM_004207 |
| | monocarboxylic acid | |
| | transporters, member 3 | |
| C14ORF58 | chromosome 14 open reading | AK000378 |
| | frame 58 | |

The aforementioned set of thirteen biomarkers, or a subset thereof, is also referred to herein as a “hypoxia profile”.

In one embodiment, the method provided herein includes determining the levels of at least five biomarkers, at least six biomarkers, at least seven biomarkers, at least eight biomarkers, at least nine biomarkers, or at least ten biomarkers, or five to thirteen, six to thirteen, seven to thirteen, eight to thirteen, nine to thirteen or ten to thirteen biomarkers selected from RRAGD, FABP5, UCHL1, GAL, PLOD, DDIT4, VEGF, ADM, ANGPTL4, NDRG1, NP, SLC16A3, and C140RF58 in a sample obtained from a subject. Biomarker expression in some instances may be normalized against the expression levels of all RNA transcripts or their expression products in the sample, or against a reference set of RNA transcripts or their expression products. The reference set as explained throughout, may be an actual sample that is tested in parallel with the sample, or may be a reference set of values from a database or stored dataset. Levels of expression, in one embodiment, are reported in number of copies, relative fluorescence value or detected fluorescence value. The level of expression of the biomarkers of the hypoxia profile together with the rTMB value and/or rate alone or in combination with other characterization methods as described herein (e.g., cancer subtype, immune subtype and/or proliferation status) as determined using the methods provided herein can be used in the methods described herein to determine whether a patient is likely to respond to angiogenesis inhibitor therapy.

In one embodiment, the levels of expression of the thirteen biomarkers (or subsets thereof, as described above, e.g., five or more, from about five to about 13), are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

In one embodiment, angiogenesis inhibitor treatments include, but are not limited to an integrin antagonist, a selectin antagonist, an adhesion molecule antagonist, an antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1), a basic fibroblast growth factor antagonist, a vascular endothelial growth factor (VEGF) modulator, a platelet derived growth factor (PDGF) modulator (e.g., a PDGF antagonist).

In one embodiment of determining whether a subject is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-1β), monocyte chemotactic protein-1 (MCP-1) and a vascular endothelial growth factor (VEGF)), as described in U.S. Pat. No. 6,524,581, incorporated by reference in its entirety herein.

The methods provided herein are also useful for determining whether a subject is likely to respond to one or more of the following angiogenesis inhibitors: interferon gamma 1β, interferon gamma 1β (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-(3 Inhibitor, transforming growth factor, P-receptor 2 oligonucleotide,VA999260, XV615 or a combination thereof

In another embodiment, a method is provided for determining whether a subject is likely to respond to one or more endogenous angiogenesis inhibitors. In a further embodiment, the endogenous angiogenesis inhibitor is endostatin, a 20 kDa C-terminal fragment derived from type XVIII collagen, angiostatin (a 38 kDa fragment of plasmin), a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN), (e.g., IFN-α, IFN-β, IFN-γ), a chemokine, e.g., a chemokine having the C-X-C motif (e.g., CXCL10, also known as interferon gamma-induced protein 10 or small inducible cytokine B10), an interleukin cytokine (e.g., IL-4, IL-12, IL-18), prothrombin, antithrombin III fragment, prolactin, the protein encoded by the TNFSF15 gene, osteopontin, maspin, canstatin, proliferin-related protein.

In one embodiment, a method for determining the likelihood of response to one or more of the following angiogenesis inhibitors is provided is angiopoietin-1, angiopoietin-2, angiostatin, endostatin, vasostatin, thrombospondin, calreticulin, platelet factor-4, TIMP, CDAI, interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1, TSP-2, interferon gamma 1β, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon a-213, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615 or a combination thereof.

In yet another embodiment, the angiogenesis inhibitor can include pazopanib (Votrient), sunitinib (Sutent), sorafenib (Nexavar), axitinib (Inlyta), ponatinib (Iclusig), vandetanib (Caprelsa), cabozantinib (Cometrig), ramucirumab (Cyramza), regorafenib (Stivarga), ziv-aflibercept (Zaltrap), motesanib, or a combination thereof. In another embodiment, the angiogenesis inhibitor is a VEGF inhibitor. In a further embodiment, the VEGF inhibitor is axitinib, cabozantinib, aflibercept, brivanib, tivozanib, ramucirumab or motesanib. In yet a further embodiment, the angiogenesis inhibitor is motesanib.

In one embodiment, the methods provided herein relate to determining a subject's likelihood of response to an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-β or PDGFR-β. In one embodiment, the PDGF antagonist is the anti-PDGF-r3 aptamer E10030, sunitinib, axitinib, sorefenib, imatinib, imatinib mesylate, nintedanib, pazopanib HC1, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

Upon making a determination of whether a patient is likely to respond to angiogenesis inhibitor therapy, or selecting a patient for angiogenesis inhibitor therapy, in one embodiment, the patient is administered the angiogenesis inhibitor. The angiogenesis in inhibitor can be any of the angiogenesis inhibitors described herein.

### Immunotherapy

In one embodiment, provided herein is a method for determining whether a cancer patient is likely to respond to immunotherapy by determining the rTMB value and/or rate alone or in combination with other characterization methods as described herein (e.g., cancer subtype, immune subtype and/or proliferation status) from a sample obtained from the patient and, based on the rTMB value and/or rate alone or in combination with other characterization methods as described herein (e.g., cancer subtype, immune subtype and/or proliferation status), assessing whether the patient is likely to respond to or may benefit from immunotherapy. In another embodiment, provided herein is a method of selecting a patient suffering from cancer for immunotherapy by determining an rTMB value and/or rate alone or in combination with other characterization methods as described herein (e.g., cancer subtype, immune subtype and/or proliferation status) of a sample from the patient and, based on the rTMB value and/or rate alone or in combination with other characterization methods as described herein (e.g., cancer subtype, immune subtype and/or proliferation status), selecting the patient for immunotherapy. The immunotherapy can be any immunotherapy provided herein. In one embodiment, the immunotherapy comprises administering one or more checkpoint inhibitors. The checkpoint inhibitors can be any checkpoint inhibitor or modulator provided herein such as, for example, a checkpoint inhibitor that targets or interacts with cytotoxic T-lymphocyte antigen 4 (CTLA4), programmed death 1 (PD-1) or its ligands (e.g., PD-L1), lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof

In another embodiment, the immunotherapeutic agent is a checkpoint inhibitor. In some cases, a method for determining the likelihood of response to one or more checkpoint inhibitors is provided. In one embodiment, the checkpoint inhibitor is a PD-1/PD-LI checkpoint inhibitor. The PD-1/PD-LI checkpoint inhibitor can be nivolumab, pembrolizumab, atezolizumab, durvalumab, lambrolizumab, or avelumab. In one embodiment, the checkpoint inhibitor is a CTLA-4 checkpoint inhibitor. The CTLA-4 checkpoint inhibitor can be ipilimumab or tremelimumab. In one embodiment, the checkpoint inhibitor is a combination of checkpoint inhibitors such as, for example, a combination of one or more PD-1/PD-LI checkpoint inhibitors used in combination with one or more CTLA-4 checkpoint inhibitors.

In one embodiment, the immunotherapeutic agent is a monoclonal antibody. In some cases, a method for determining the likelihood of response to one or more monoclonal antibodies is provided. The monoclonal antibody can be directed against tumor cells or directed against tumor products. The monoclonal antibody can be panitumumab, matuzumab, necitumunab, trastuzumab, amatuximab, bevacizumab, ramucirumab, bavituximab, patritumab, rilotumumab, cetuximab, immu-132, or demcizumab.

In yet another embodiment, the immunotherapeutic agent is a therapeutic vaccine. In some cases, a method for determining the likelihood of response to one or more therapeutic vaccines is provided. The therapeutic vaccine can be a peptide or tumor cell vaccine. The vaccine can target MAGE-3 antigens, NY-ESO-1 antigens, p53 antigens, survivin antigens, or MUC1 antigens. The therapeutic cancer vaccine can be GVAX (GM-CSF gene-transfected tumor cell vaccine), belagenpumatucel-L (allogeneic tumor cell vaccine made with four irradiated NSCLC cell lines modified with TGF-beta2 antisense plasmid), MAGE-A3 vaccine (composed of MAGE-A3 protein and adjuvant AS15), (1)-BLP-25 anti-MUC-1 (targets MUC-1 expressed on tumor cells), CimaVax EGF (vaccine composed of human recombinant Epidermal Growth Factor (EGF) conjugated to a carrier protein), WT1 peptide vaccine (composed of four Wilms' tumor suppressor gene analogue peptides), CRS-207 (live-attenuated Listeria monocytogenes vector encoding human mesothelin), Bec2/BCG (induces anti-GD3 antibodies), GV1001 (targets the human telomerase reverse transcriptase), TG4010 (targets the MUC1 antigen), racotumomab (anti-idiotypic antibody which mimicks the NGcGM3 ganglioside that is expressed on multiple human cancers), tecemotide (liposomal BLP25; liposome-based vaccine made from tandem repeat region of MUC1) or DRibbles (a vaccine made from nine cancer antigens plus TLR adjuvants).

In one embodiment, the immunotherapeutic agent is a biological response modifier. In some cases, a method for determining the likelihood of response to one or more biological response modifiers is provided. The biological response modifier can trigger inflammation such as, for example, PF-3512676 (CpG 7909) (a toll-like receptor 9 agonist), CpG-ODN 2006 (downregulates Tregs), Bacillus Calmette-Guerin (BCG), mycobacterium vaccae (SRL172) (nonspecific immune stimulants now often tested as adjuvants). The biological response modifier can be cytokine therapy such as, for example, IL-2+ tumor necrosis factor alpha (TNF-alpha) or interferon alpha (induces T-cell proliferation), interferon gamma (induces tumor cell apoptosis), or Mda-7 (IL-24) (Mda-7/IL-24 induces tumor cell apoptosis and inhibits tumor angiogenesis). The biological response modifier can be a colony-stimulating factor such as, for example granulocyte colony-stimulating factor. The biological response modifier can be a multi-modal effector such as, for example, multi-target VEGFR: thalidomide and analogues such as lenalidomide and pomalidomide, cyclophosphamide, cyclosporine, denileukin diftitox, talactoferrin, trabecetedin or all-trans-retinmoic acid.

In one embodiment, the immunotherapy is cellular immunotherapy. In some cases, a method for determining the likelihood of response to one or more cellular therapeutic agents. The cellular immunotherapeutic agent can be dendritic cells (DCs) (ex vivo generated DC-vaccines loaded with tumor antigens), T-cells (ex vivo generated lymphokine-activated killer cells; cytokine-induce killer cells; activated T-cells; gamma delta T-cells), or natural killer cells.

### Radiotherapy

In one embodiment, provided herein is a method for determining whether a patient is likely to respond to radiotherapy by determining the rTMB value and/or rate alone or in combination with other characterization methods as described herein (e.g., cancer subtype, immune subtype and/or proliferation status) of a sample obtained from the patient and, based on the rTMB value and/or rate alone or in combination with other characterization methods as described herein (e.g., cancer subtype, immune subtype and/or proliferation status), assessing whether the patient is likely to respond to or benefit from radiotherapy. In another embodiment, provided herein is a method of selecting a patient suffering from cancer for radiotherapy by determining an rTMB value and/or rate alone or in combination with other characterization methods as described herein (e.g., cancer subtype, immune subtype and/or proliferation status) of a sample from the patient and, based on the rTMB value and/or rate alone or in combination with other characterization methods as described herein (e.g., cancer subtype, immune subtype and/or proliferation status), selecting the patient for radiotherapy.

In some embodiments, the radiotherapy can include but are not limited to proton therapy and external-beam radiation therapy. In some embodiments, the radiotherapy can include any types or forms of treatment that is suitable for patients with specific types of cancer. In some embodiments, the surgery can include laser technology, excision, dissection, and reconstructive surgery.

In some embodiments, an patient with a specific type of cancer can have or display resistance to radiotherapy. Radiotherapy resistance in any cancer of subtype thereof can be determined by measuring or detecting the expression levels of one or more genes known in the art and/or provided herein associated with or related to the presence of radiotherapy resistance. Genes associated with radiotherapy resistance can include NFE2L2, KEAP1 and CUL3. In some embodiments, radiotherapy resistance can be associated with the alterations of KEAP1(Kelch-like ECH-associated protein 1)/NRF2 (nuclear factor E2-related factor 2) pathway. Association of a particular gene to radiotherapy resistance can be determined by examining expression of said gene in one or more patients known to be radiotherapy non-responders and comparing expression of said gene in one or more patients known to be radiotherapy responders.

Surgical Intervention

In one embodiment, provided herein is a method for determining whether a HNSCC cancer patient is likely to respond to surgical intervention by determining the rTMB value and/or rate alone or in combination with other characterization methods as described herein (e.g., cancer subtype, immune subtype and/or proliferation status)of a sample obtained from the patient and, based on the rTMB value and/or rate alone or in combination with other characterization methods as described herein (e.g., cancer subtype, immune subtype and/or proliferation status), assessing whether the patient is likely to respond to or benefit from surgery. In another embodiment, provided herein is a method of selecting a patient suffering from cancer for surgery by determining an rTMB value and/or rate alone or in combination with other characterization methods as described herein (e.g., cancer subtype, immune subtype and/or proliferation status) of a sample from the patient and, based on the rTMB value and/or rate alone or in combination with other characterization methods as described herein (e.g., cancer subtype, immune subtype and/or proliferation status), selecting the patient for surgery.

In some embodiments, surgery approaches for use herein can include but are not limited to minimally invasive or endoscopic head and neck surgery (eHNS), Transoral Robotic Surgery (TORS), Transoral Laser Microsurgery (TLM), Endoscopic Thyroid and Neck Surgery, Robotic Thyroidectomy, Minimally Invasive Video-Assisted Thyroidectomy (MIVAT), and Endoscopic Skull Base Tumor Surgery. In some embodiments, the surgery can include any types of surgical treatment that is suitable for HNSCC patients. In one embodiment, the suitable treatment is surgery.

### EXAMPLES

The present invention is further illustrated by reference to the following Examples. However, it should be noted that these Examples, like the embodiments described above, are illustrative and are not to be construed as restricting the scope of the invention in any way.

### Example 1

### Development and Validation of Method for Calculating TMB Using RNA-Seq Data

### Objective

This example describes the generation of a method for determining tumor mutational burden (TMB) value and rate from RNA sequencing data (e.g., paired-end RNA-seq data). The method employed an algorithm developed herein that was used to analyze the RNA sequencing data obtained from transcriptome profiling studies on tumor samples in order to determine the TMB of said samples. Given that TMB has been shown to predict response to immunotherapy treatments including PD-1 and PD-Ll inhibitors, results of this type of RNA-seq TMB analyses may also be useful for informing immunotherapeutic response. Further, the RNA-seq TMB analyses provided in this example may represent a cost-effective alternative to gold standard DNA based TMB rate determination that can be performed on tumor samples alone rather than using both tumor samples and matched normal samples, which is often done when calculating TMB using DNA sequencing data.

### Methods and Results

In order to develop an algorithm for use in the method for determining TMB value and TMB rate from RNA, paired end RNA-seq data from the lung adenocarcinoma (LUAD) dataset (n=105) from TCGA was downloaded from the NIH National Cancer Institute GDC data portal (https://portal.gdc.cancer.gov). In particular, ⅔ of the LUAD RNA-seq TCGA dataset (n=70) was used as a training set for determining algorithm parameters (e.g., reads ratio threshold and sequencing coverage for TMB rate calculations), while the remaining 1/3 of the LUAD RNA-seq dataset (n=35) was used to test the resultant algorithm (see details below). The desired output of the algorithm was a TMB rate from the RNA-seq data that correlated well with the TMB calculations obtained from a gold standard TMB method⁸.

As shown schematically in FIG. 1, the algorithm as implemented on a computer comprised a series of sequential steps represented as blocks 1-10 in FIG. 1. Given that some of the steps of the algorithm required the RNA-seq data to be in text format, the compressed BAM files of RNA-seq data obtained from TCGA for the LUAD RNA-seq dataset were converted from the compressed BAM file format to a text-based fastq format using Bedtools (version 2.27.1) bamtofastql as necessary prior to running the data through the algorithm.

As shown in FIG. 1, following conversion to fastq format, the RNA seq data from the training set (i.e., LUAD RNA-seq TCGA dataset (n=70)) was processed through the algorithm which comprised: aligning the fastq converted RNA-seq data to a human reference genome (i.e., the GRCh38v22 (10.2014 release hg38) version of the GRCh38 human genome reference) using STAR software' (version 2.5.3a; block 1 of FIG. 1), sorting and indexing reads using Sambamba software' (version v0.6..7 linux; block 2 of FIG. 1), re-aligning reads using ABRA2⁴ (version abra2-2.14; block 3 of FIG. 1), removing adjacent SNP/Indels using SAMtools⁵ (version 1.6-1-gdd8cab5; block 4 of FIG. 1), determining a normalization factor for TMA rate calculations using Picard CollectHsMetrics and calling variants using STRELKA2⁶ (version strelka-2.9.0; block 5 of FIG. 1), removing low-confidence calls and non-canonical chromosomes (i.e. “chrUn”, “random”, “decoy”, “chrM”, “chrY”) using STRELKA2 default filters (block 6 of FIG. 1), and annotating the remaining SNPs using Variant Effect Prediction⁷ (VEP; version ensembl-vep 91.3 (cached, offline version); block 7 of FIG. 1) in order to facilitate further filtering of the remaining SNPs. The annotation included SNP location, alleles, allele counts, missense status, dbSNP status and gene symbol. The annotated SNPs were then subjected to a series of filtering steps. (i.e., blocks 8-10 of FIG. 1). The filtering and prioritization steps included: (1) removing SNPs in HLA and IG genes (gene symbol starts with “HLA” or “IG”); (2) removing SNPs with fewer than 25 total reads; (3) removing SNPs in dbSNP (dbSNP version 150, which is used by VEP version 91); (4) removing SNPs not called “missense variant” by VEP; (5) removing SNPs having a reads ratio not consistent with somatic mutation (i.e., SNPs with read ratios (reference allele reads/total reads) near 0, ½, or 1) and (6) converting the TMB value obtained from the preceding algorithm steps into a TMB rate. by normalizing the value to a transcriptome targeted region with high coverage (i.e., sequencing depth).

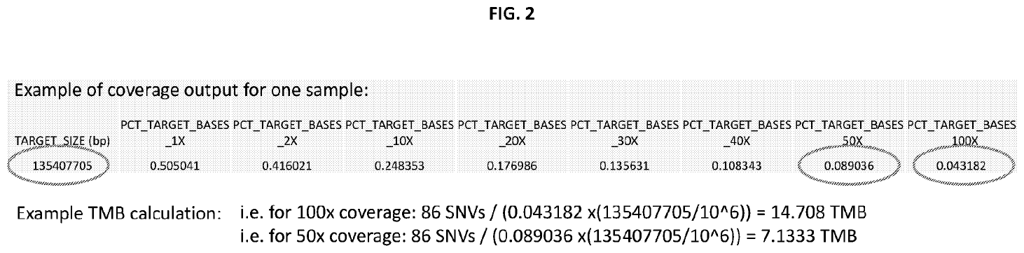

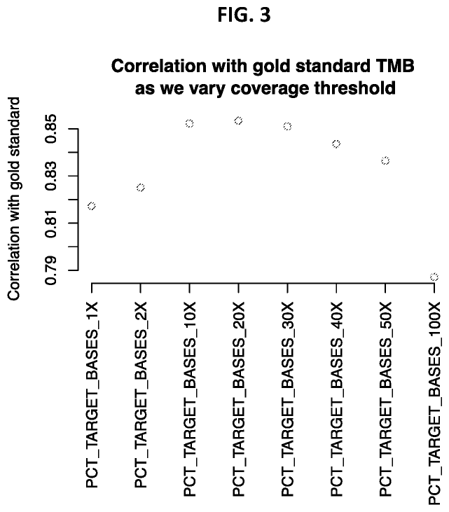

With regards to filtering and prioritization step (6), a TMB rate was calculated for each of the other filtering steps described above in order to determine the necessity of each respective step in the algorithm (described further below). The number of SNPs remaining following each of the filtering steps 1-5 above represented a TMB value. In order to calculate the TMB rate at each of the filtering steps, the TMB value at each step was normalized to a transcriptome targeted region with high coverage to yield the number of SNPs per mb. More specifically, the normalization equaled the TMB value (i.e., SNP counts)/(percent of target with a specific coverage (e.g., 1×, 10×, 20×, 50×, 100×)) X (genome target size per mb). The total possible genome target size used for this calculation was based on all exons with +/−10bp of flanking sequence and was found to be 135407705 bps. In order to determine the optimal coverage for the TMB rate calculation, Picard CollectHsMetrics was used as depicted in block 4 of FIG. 1 on the training set in order to get coverage output values for each sample from the training set. FIG. 2 represents coverage output for one sample and example TMB rate calculations for specific coverage outputs. Ultimately, using the training data set and correlation analysis with the gold standard TAM⁸ for LUAD, it was found that 20× coverage in the target region size estimate rather than the additional levels of coverage tested (e.g., 1×, 10×, 20×, 30×, 40×, 50× or 100×) maximized rank correlation with the gold standard TMB (see FIG. 3).

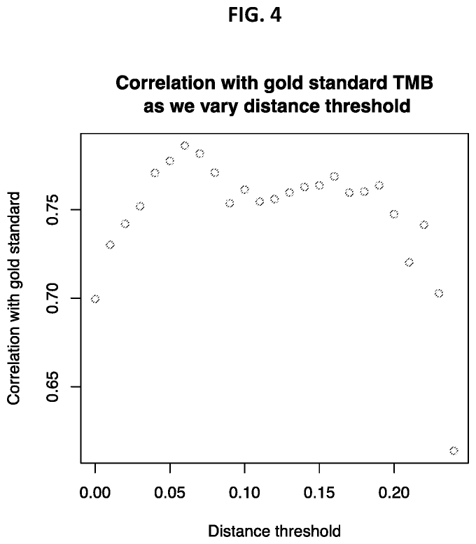

The other parameter for which the training set (n=70 LUAD) was used to determine the reads ratio threshold used in filtering step 5. With regards to the reads ratio threshold, the goal was to remove SNPs from the TMB calculation when the reference allele reads and total reads were inconsistent with somatic mutation. Namely, SNPs having a reads ratio (reference allele reads divided by total reads) close to 0, ½, or 1 were considered inconsistent. Using the training set (n=70 LUAD), it was found that requiring the reads ratio to be at least 0.06 in value away from 0, 1/2, and 1 maximized the rank correlation with gold standard TMB (see FIG. 4).

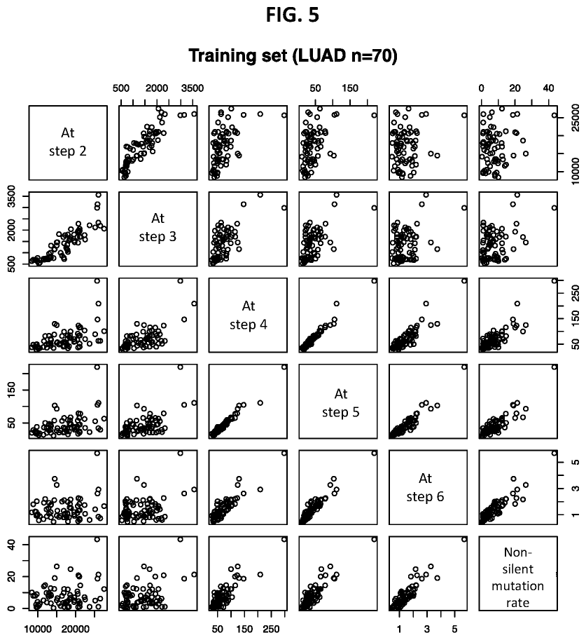

As mentioned above, the algorithm comprises a series of filtering steps (i.e., represented by blocks 8-10 in FIG. 1). These filtering steps were introduced in order to optimize said algorithm for calculating TMB rate from RNA sequencing data. Once the TMB rate was calculated for each filtering step as described above, a correlation analysis with the gold standard TMB rate for the LUAD dataset as found in Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018, The immune landscape of cancer. Immunity, 48(4), pp. 812-830, was performed for each filtering step. As shown in FIG. 5, starting following filtering step 1 (i.e., all algorithm steps up to and including exclusion of SNPs in HLA and IG genes as described above; ‘at step 2’ in FIG. 5) and working progressively through step 2 (i.e., all algorithm steps up to and including exclusion of SNPs with fewer than 25 total reads as described above; ‘at step 3’ in FIG. 5), step 3 (i.e., all algorithm steps up to and including exclusion of SNPs in dbSNP as described above; ‘at step 4’ in FIG. 5), step 4 (i.e., all algorithm steps up to and including exclusion of SNPs not annotated “missense variant” as described above; ‘at step 5’ in FIG. 5) step 5 (i.e., all algorithm steps up to and including exclusion of SNPs using reads ratio threshold=0.06; ‘at step 6’ in FIG. 5) and step 6 (i.e., calculating TMB rate using coverage value=20× and incorporating all of the preceding filtering steps), rank correlations were determined between the TMB rate for each respective step with the gold standard TMB rate as found in the supplemental files of Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018, The immune landscape of cancer. Immunity, 48(4), pp.812-830. As can be seen in FIG. 5, the rank correlation between RNA-seq based TMB rates with gold standard DNA-seq TMB rates increased with the progressive introduction of each of the detailed filtering steps.

### Validation

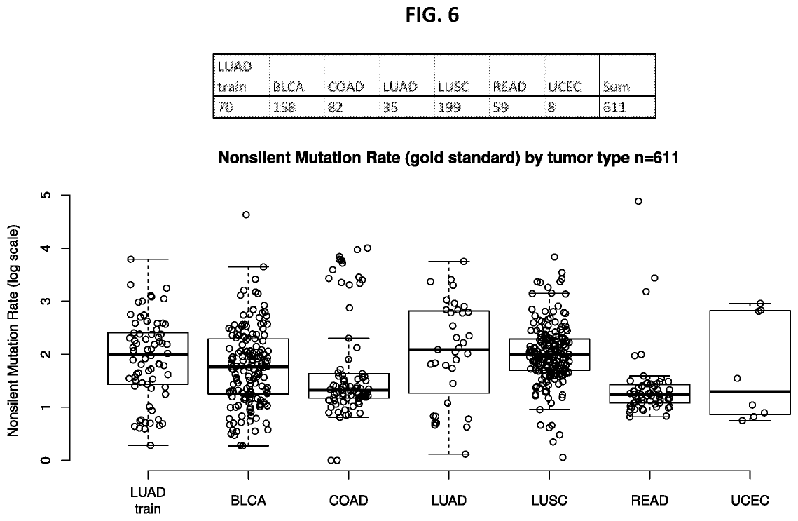

In order to validate the algorithm developed herein, paired-end RNAseq BAM files (HiSeq) were downloaded from TCGA (https://portal.gdc.cancer.gov/) for primary solid tumor samples from the following TCGA studies: BLCA, COAD, LUAD, LUSC, READ, and UCEC and converted to fastq file format as necessary as provided herein. These studies were chosen because, in addition to having TCGA RNA-seq datasets, each possessed samples that had DNA-based Tumor Mutation Burden (TMB) values found in the supplemental data files of Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018. The immune landscape of cancer. Immunity, 48(4), pp. 812-830. A total of n=611 samples were downloaded. It is noted that, as described above, 2/3 of the LUAD data (n=70) was used as a training set, while the remaining ⅓ of the LUAD data (n=35) was used as a testing set along with the datasets from the other 5 studies described above. As a reference, the non-silent mutation rate for each sample from each tumor type as determined from DNA sequencing data (see supplemental data in Thorsson et al.⁸) used the gold standard TMB method is shown in FIG. 6. The legend within FIG. 6 details the sample size by tumor type used to calculate non-silent tumor rate by the gold standard TMB method⁸.

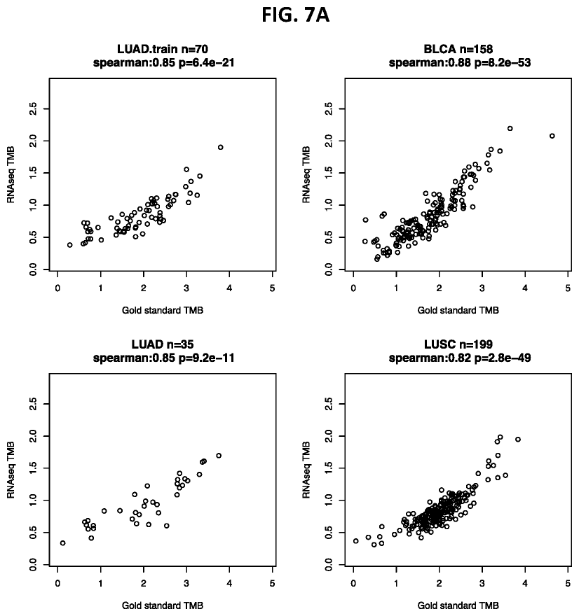

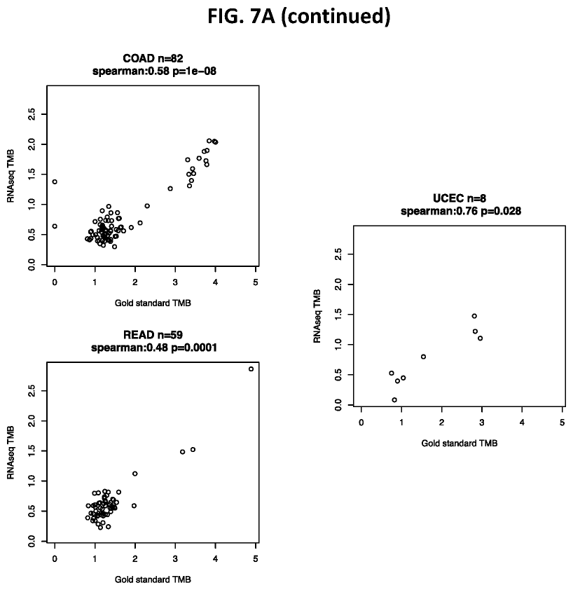

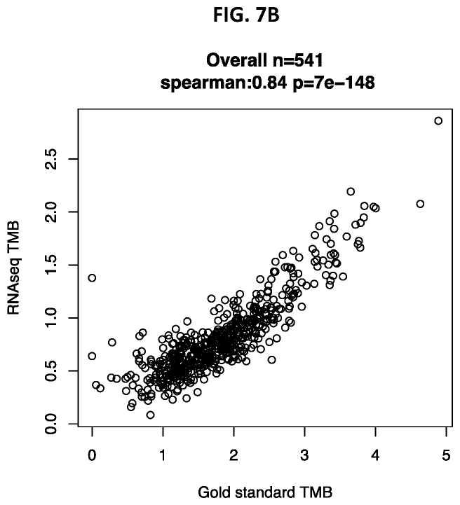

The algorithm developed and described herein was subsequently applied to the n=611 samples from the 6 TCGA studies described above and correlations with gold standard TMB (FIGS. 7A-7B) were examined, separately in each tumor type (FIG. 7A) and in the pooled data (FIG. 7B) excluding the training set. As shown in Table 2 and FIG. 7A, the spearman correlation coefficient in the LUAD training set was 0.85. In other data sets, the correlation ranged from 0.48 in the READ dataset, which has uniformly low TMB relative to other tumor types, to 0.88 in BLCA, which has tumors with highly variable TMB (see Table 2 and FIG. 7A). Correlation test p-values were highly significant overall and modest in UCEC due to small sample size (n=8). In the pooled data, the spearman correlation coefficient was 0.84.

TABLE 2
Correlations with gold standard TMB by data set
(“overall” excludes training).

| | n | spearman | p | pearson |
|---|---|---|---|---|
| LUAD.train | 70 | 0.85 | 6.40E−21 | 0.91 |
| BLCA | 158 | 0.88 | 8.20E−53 | 0.81 |
| COAD | 82 | 0.58 | 1.00E−08 | 0.96 |
| LUAD | 35 | 0.85 | 9.20E−11 | 0.92 |
| LUSC | 199 | 0.82 | 2.80E−49 | 0.9 |
| READ | 59 | 0.48 | 0.0001 | 0.99 |
| UCEC | 8 | 0.76 | 0.028 | 0.89 |
| overall | 541 | 0.84 | 7.00E−148 | 0.92 |

Note Pearson correlation coefficients were calculated using the RNAseq-derived TMB and gold standard values prior to log transformation for the plots. The extreme Pearson correlation in the READ data set is driven by an outlier. When that sample is excluded, Pearson correlation=0.88

Conclusions

Overall, it has been shown that transcriptomic profiling data can be successfully used to determine the TMB value and rate in tumor samples from a variety of different types of cancer. In contrast to assessing TMB through the use of DNA sequencing data obtained either through whole exome sequencing or sequencing of a subset of the genome or exome, RNA-based TMB analysis provides an estimate of the amount and/or level of mutations found in the transcriptome of a tumor and can take into account both mutations found at the DNA level (i.e., genome and/or exome) and at the RNA level (e.g., mutations that arise as a result of RNA editing). As such, RNA-based TMB analysis may provide a more accurate representation of the number and/or level of neoantigens present within a tumor, which may aid in informing on patient-specific cancer therapies such as, for example, cancer immunotherapies. Further, RNA-based TMB (rTMB) may also aid in the development of next-generation immunotherapies by providing tumor relevant neoantigens.

### INCORPORATION BY REFERENCE

The following references are referenced throughout the text and are incorporated by reference in their entireties for all purposes.

1. Quinlan A R, et al. BEDTools: a flexible suite of utilities for comparing genomic features. Bioinformatics. 2010 Mar. 15; 26(6): 841-842.

2. Dobin A, Davis C A, Schlesinger F, Drenkow J, Zaleski C, Jha S, Batut P, Chaisson M, Gingeras T R. “STAR: ultrafast universal RNA-seq aligner”. Bioinformatics. 2013 Jan. 1; 29(1):15-21. doi: 10.1093/bioinformatics/bts635. Epub 2012 Oct. 25.

3. A. Tarasov, A. J. Vilella, E. Cuppen, I. J. Nijman, and P. Prins. Sambamba: fast processing of NGS alignment formats. Bioinformatics, 2015.

4. Mose L E, Wilkerson M D, Hayes D N, Perou C M, Parker J S. ABRA: improved coding indel detection via assembly-based realignment. Bioinformatics. 2014; 30:2813-2815. doi: 10.1093/bioinformatics/btu376.

5. Li, H.; Handsaker, B.; Wysoker, A.; Fennell, T.; Ruan, J.; Homer, N.; Marth, G.; Abecasis, G.; Durbin, R.; 1000 Genome Project Data Processing Subgroup (2009). “The Sequence Alignment/Map format and SAMtools”. Bioinformatics. 25 (16): 2078-2079.

6. Kim S. et al., Strelka2: fast and accurate calling of germline and somatic variants. Nature Methods, volume 15, pages591-594 (2018).

7. McLaren W, Gil L, Hunt S E, Riat H S, Ritchie G R, Thormann A, Flicek P, Cunningham F. The Ensembl Variant Effect Predictor. Genome Biology Jun 6;17(1):122. (2016).

8. Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018. The immune landscape of cancer. Immunity, 48(4), pp. 812-830.

Further Numbered Embodiments of the Disclosure

Other subject matter contemplated by the present disclosure is set out in the following numbered embodiments:

1. A method of analyzing a tumor sample for a mutation load, comprising: detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germline variants;

annotating the plurality of detected variants with annotation information from one or more population databases, wherein the population databases include information associated with variants in a population, wherein the annotation information includes missense status and germline alteration status associated with a given variant, thereby generating a plurality of annotated variants;

filtering the plurality of annotated variants, wherein the filtering applies a rule set to the annotated variants to retain the detected variants that are non-synonymous somatic single nucleotide variants (SNVs), the rule set comprises:

(i) removing SNVs corresponding to SNPs in a database of germline alterations; and

(ii) removing SNVs not annotated as missense variants, wherein the filtering produces identified non-synonymous somatic SNVs;

counting the identified non-synonymous somatic SNVs to give a tumor mutation value; determining a number of bases in the genomic regions targeted by the transcriptomic profile in the tumor sample genome; and

calculating a number of non-synonymous somatic SNVs per megabase by dividing the tumor mutation value by the number of bases in the genomic regions targeted by the transcriptomic profile to produce the mutation load.

2. The method of embodiment 1, wherein the population databases include one or more of a 1000 genomes database, Ensembl variation databases, COSMIC, Human Gene Mutation Database dbSNP, and an Exome Aggregation Consortium (ExAC) database.

3. The method of embodiment 1 or 2, wherein the database of germline alterations in the dbSNP database.

4. The method of embodiment 1, wherein the rule set further comprises removing the SNVs present in HLA and Ig genes and removing the SNVs with fewer than 25 total reads prior to (i).

5. The method of any one of embodiments 1-4, wherein the rule set further comprises removing SNPs having a reads ratio inconsistent with somatic mutation following step (ii), wherein the reads ratio equals reference allele reads/total reads.

6. The method of embodiment 1, wherein the number of bases in the genomic regions targeted by the transcriptomic profile used to divide the tumor mutation value is multiplied by the percentage of bases with a desired sequencing depth.

7. The method of embodiment 6, wherein the desired sequencing depth is 20X.

8. The method of any one of the above embodiments, wherein the genomic regions targeted by the transcriptomic profile are exons.

9. The method of any one of the above embodiments, wherein the detecting variants is configured by variant caller parameters, the variant caller parameters including a minimum allele frequency parameter, a strand bias parameter and a data quality stringency parameter.

10. The method of any one of the above embodiments, wherein, prior to detecting variants, the method comprises aligning the nucleic acid sequence reads obtained from the transcriptomic profiling to a human reference genome; sorting and indexing; re-aligning to remove alignment errors and reference bias; and removing adjacent SNVs and indels.

11. The method of embodiment 10, wherein the aligning the nucleic acid sequence reads obtained from the transcriptomic profiling to the human reference genome is performed with a spliced mapper.

12. A system for analyzing a tumor sample genome for a mutation load, comprising a processor and a data store communicatively connected with the processor, the processor configured to perform the steps including:

detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germ-line variants;

annotating the plurality of detected variants with annotation information from one or more population databases, wherein the population databases include information associated with variants in a population, wherein the annotation information includes missense status and germline alteration status associated with a given variant, thereby generating a plurality of annotated variants;

filtering the plurality of annotated variants, wherein the filtering applies a rule set to the annotated variants to retain the detected variants that are non-synonymous somatic single nucleotide variants (SNVs), the rule set comprises:

(i) removing SNVs corresponding to SNPs in a database of germline alterations; and

(ii) removing SNVs not annotated as missense variants, wherein the filtering produces identified non-synonymous somatic SNVs;

counting the identified non-synonymous somatic SNVs to give a tumor mutation value; determining a number of bases in the genomic regions targeted by the transcriptomic profile in the tumor sample genome; and

calculating a number of non-synonymous somatic SNVs per megabase by dividing the tumor mutation value by the number of bases in the genomic regions targeted by the transcriptomic profile to produce the mutation load.

13. The system of embodiment 12, wherein the population databases include one or more of a 1000 genomes database, Ensembl variation databases, COSMIC, Human Gene Mutation Database dbSNP, and an Exome Aggregation Consortium (ExAC) database.

14. The system of embodiment 12 or 13, wherein the database of germline alterations in the dbSNP database.

15. The method of embodiment 12, wherein the rule set further comprises removing the SNVs present in HLA and Ig genes and removing the SNVs with fewer than 25 total reads prior to (i).

16. The system of any one of embodiments 12-15, wherein the rule set further comprises removing SNPs having a reads ratio inconsistent with somatic mutation following step (ii), wherein the reads ratio equals reference allele reads/total reads.

17. The system of embodiment 12, wherein the number of bases in the genomic regions targeted by the transcriptomic profile used to divide the tumor mutation value is multiplied by the percentage of bases with a desired sequencing depth.

18. The system of embodiment 17, wherein the desired sequencing depth is 20X.

19. The system of any one of embodiments 12-18, wherein the genomic regions targeted by the transcriptomic profile are exons.

20. The system of any one of embodiments 12-19, wherein the detecting variants is configured by variant caller parameters, the variant caller parameters including a minimum allele frequency parameter, a strand bias parameter and a data quality stringency parameter.

21. The system of any one of embodiments 12-20, wherein, prior to detecting variants, the method comprises aligning the nucleic acid sequence reads obtained from the transcriptomic profiling to a human reference genome; sorting and indexing; re-aligning to remove alignment errors and reference bias; and removing adjacent SNVs and indels.

22. The system of embodiment 21, wherein the aligning the nucleic acid sequence reads obtained from the transcriptomic profiling to the human reference genome is performed with a spliced mapper.

23. A non-transitory machine-readable storage medium comprising instructions which, when executed by a processor, cause the processor to perform a method analyzing a tumor sample genome for a mutation load, comprising:

detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germ-line variants;

annotating the plurality of detected variants with annotation information from one or more population databases, wherein the population databases include information associated with variants in a population, wherein the annotation information includes missense status and germline alteration status associated with a given variant, thereby generating a plurality of annotated variants;

filtering the plurality of annotated variants, wherein the filtering applies a rule set to the annotated variants to retain the detected variants that are non-synonymous somatic single nucleotide variants (SNVs), the rule set comprises:

(i) removing SNVs corresponding to SNPs in a database of germline alterations; and

(ii) removing SNVs not annotated as missense variants, wherein the filtering produces identified non-synonymous somatic SNVs;

counting the identified non-synonymous somatic SNVs to give a tumor mutation value; determining a number of bases in the genomic regions targeted by the transcriptomic profile in the tumor sample genome; and

calculating a number of non-synonymous somatic SNVs per megabase by dividing the tumor mutation value by the number of bases in the genomic regions targeted by the transcriptomic profile to produce the mutation load.

24. A method of identifying an individual having a cancer who may benefit from a cancer therapy, the method comprising determining a tumor mutational burden (TMB) rate using RNA sequencing data obtained from a tumor sample from the individual, wherein a TMB rate from the tumor sample that is at or above a reference TMB rate identifies the individual as one who may benefit from the cancer therapy.

25. A method for selecting a cancer therapy for an individual having a cancer, the method comprising determining a TMB rate using RNA sequencing data from a tumor sample from the individual, wherein a TMB rate from the tumor sample that is at or above a reference TMB rate identifies the individual as one who may benefit from the cancer therapy.

26. The method of embodiment 24 or 25, wherein the TMB rate determined from the tumor sample is at or above the reference TMB rate, and the method further comprises administering to the individual an effective amount of the cancer therapy.

27. The method of embodiment 24 or 25, wherein the TMB rate determined from the tumor sample is below the reference TMB rate.

28. A method of treating an individual having a cancer, the method comprising:
(a) determining a TMB rate from a tumor sample obtained from the individual, wherein the TMB rate from the tumor sample is at or above a reference TMB rate, and wherein the TMB rate is calculated from RNA sequencing data; and
(b) administering a cancer therapy to the individual.

29. The method of any one of embodiments 24-28, wherein the reference TMB rate is a pre-assigned TMB rate.

30. The method of any one of embodiments 24-29, wherein the reference TMB rate is between about 2 and about 5 mutations per megabase (mut/Mb).

31. The method of any one of embodiments 24-30, wherein the TMB rate using RNA sequencing data reflects a rate of non-synonymous somatic mutations.

32. The method of embodiment 31, wherein the rate of non-synonymous somatic mutations represents a rate of candidate neoantigens.

33. The method of embodiment 31 or 32, wherein the non-synonymous somatic mutations comprise mutations that have arisen due to RNA editing.

34. The method of any one of embodiments 24-33, wherein the cancer is a cervical kidney renal papillary cell carcinoma (KIRP); breast invasive carcinoma (BRCA); thyroid cancer (THCA); bladder carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head-neck squamous cell carcinoma (HNSC); uterine corpus endometrial carcinoma (UCEC); glioblastoma multiforme (GBM); esophageal carcinoma (ESCA); stomach adenocarcinoma (STAD); ovarian cancer (OV); rectum adenocarcinoma (READ) or lung squamous cell carcinoma (LUSC).

35. The method of embodiment 33, wherein the cancer is lung adenocarcinoma (LUAD); colon adenocarcinoma (COAD), breast invasive carcinoma (BRCA), uterine corpus endometrial carcinoma (UCEC), rectum adenocarcinoma (READ) or lung squamous cell carcinoma (LUSC).

36. The method of any one of embodiments 24-35, wherein the cancer therapy is selected from surgical intervention, radiotherapy, one or more chemotherapeutic agents, one or more PARP inhibitors, and one or more immunotherapeutic agents.

37. The method of embodiment 36, wherein the one or more immunotherapeutic agents is an immune checkpoint modulator.

38. The method of embodiment 37, wherein the immune checkpoint modulator interacts with cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof

39. The method of embodiment 37 or 38, wherein the immune checkpoint modulator is an antibody agent.

40. The method of embodiment 39, wherein the antibody agent is or comprises a monoclonal antibody or antigen binding fragment thereof.

41. The method of any one of embodiments 24-40, wherein the determining the TMB rate using RNA sequencing data comprises:
detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germline variants;
annotating the plurality of detected variants with annotation information from one or more population databases, wherein the population databases include information associated with variants in a population, wherein the annotation information includes missense status and germline alteration status associated with a given variant, thereby generating a plurality of annotated variants;
filtering the plurality of annotated variants, wherein the filtering applies a rule set to the annotated variants to retain the detected variants that are non-synonymous somatic single nucleotide variants (SNVs), the rule set comprises:
(i) removing SNVs corresponding to SNPs in a database of germline alterations; and
(ii) removing SNVs not annotated as missense variants, wherein the filtering produces identified non-synonymous somatic SNVs;
counting the identified non-synonymous somatic SNVs to give a tumor mutation value; determining a number of bases in the genomic regions targeted by the transcriptomic profile in the tumor sample genome; and
calculating a number of non-synonymous somatic SNVs per megabase by dividing the tumor mutation value by the number of bases in the genomic regions targeted by the transcriptomic profile to produce the mutation load.

42. The method of embodiment 41, wherein the population databases include one or more of a 1000 genomes database, Ensembl variation databases, COSMIC, Human Gene Mutation Database dbSNP, and an Exome Aggregation Consortium (ExAC) database.

43. The method of embodiment 41 or 42, wherein the database of germline alterations in the dbSNP database.

44. The method of embodiment 41, wherein the rule set further comprises removing the SNVs present in HLA and Ig genes and removing the SNVs with fewer than 25 total reads prior to (i).

45. The method of any one of embodiments 41-44, wherein the rule set further comprises removing SNPs having a reads ratio inconsistent with somatic mutation following step (ii), wherein the reads ratio equals reference allele reads/total reads.

46. The method of embodiment 41, wherein the number of bases in the genomic regions targeted by the transcriptomic profile used to divide the tumor mutation value is multiplied by the percentage of bases with a desired sequencing depth.

47. The method of embodiment 46, wherein the desired sequencing depth is 20X.

48. The method of any one of embodiments 41-47, wherein the genomic regions targeted by the transcriptomic profile are exons.

49. The method of any one of embodiments 41-48, wherein the detecting variants is configured by variant caller parameters, the variant caller parameters including a minimum allele frequency parameter, a strand bias parameter and a data quality stringency parameter.

50. The method of any one of embodiments 41-49, wherein, prior to detecting variants, the method comprises aligning the nucleic acid sequence reads obtained from the transcriptomic profiling to a human reference genome; sorting and indexing; re-aligning to remove alignment errors and reference bias; and removing adjacent SNVs and indels.

51. The method of embodiment 50, wherein the aligning the nucleic acid sequence reads obtained from the transcriptomic profiling to the human reference genome is performed with a spliced mapper.

52. The method of embodiment 50 or 51, wherein the human reference genome is the GRCh38 human reference genome.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of analyzing a tumor sample for a mutation load, comprising:
detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germline variants;
annotating the plurality of detected variants with annotation information from one or more population databases, wherein the population databases include information associated with variants in a population, wherein the annotation information includes missense status and germline alteration status associated with a given variant, thereby generating a plurality of annotated variants;
filtering the plurality of annotated variants, wherein the filtering applies a rule set to the annotated variants to retain the detected variants that are non-synonymous somatic single nucleotide variants (SNVs), the rule set comprises:
(i) removing SNVs corresponding to SNPs in a database of germline alterations; and
(ii) removing SNVs not annotated as missense variants, wherein the filtering
produces identified non-synonymous somatic SNVs;
counting the identified non-synonymous somatic SNVs to give a tumor mutation value;
determining a number of bases in the genomic regions targeted by the transcriptomic profile in the tumor sample genome; and
calculating a number of non-synonymous somatic SNVs per megabase by dividing the tumor mutation value by the number of bases in the genomic regions targeted by the transcriptomic profile to produce the mutation load.

2. The method of claim 1, wherein the population databases include one or more of a 1000 genomes database, Ensembl variation databases, COSMIC, Human Gene Mutation Database dbSNP, and an Exome Aggregation Consortium (ExAC) database.

3. The method of claim 1 or 2, wherein the database of germline alterations in the dbSNP database.

4. The method of claim 1, wherein the rule set further comprises removing the SNVs present in HLA and Ig genes and removing the SNVs with fewer than 25 total reads prior to (i).

5. The method of claim 1, wherein the rule set further comprises removing SNPs having a reads ratio inconsistent with somatic mutation following step (ii), wherein the reads ratio equals reference allele reads/total reads.

6. The method of claim 1, wherein the number of bases in the genomic regions targeted by the transcriptomic profile used to divide the tumor mutation value is multiplied by the percentage of bases with a desired sequencing depth.

7. The method of claim 6, wherein the desired sequencing depth is 20×.

8. The method of claim 1, wherein the genomic regions targeted by the transcriptomic profile are exons.

9. The method of claim 1, wherein the detecting variants is configured by variant caller parameters, the variant caller parameters including a minimum allele frequency parameter, a strand bias parameter and a data quality stringency parameter.

10. The method of claim 1, wherein, prior to detecting variants, the method comprises aligning the nucleic acid sequence reads obtained from the transcriptomic profiling to a human reference genome, sorting and indexing; re-aligning to remove alignment errors and reference bias; and removing adjacent SNVs and indels.

11. The method of claim 10, wherein the aligning the nucleic acid sequence reads obtained from the transcriptomic profiling to the human reference genome is performed with a spliced mapper.

12. A system for analyzing a tumor sample genome for a mutation load, comprising a processor and a data store communicatively connected with the processor, the processor configured to perform the steps including:
detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germ-line variants;
annotating the plurality of detected variants with annotation information from one or more population databases, wherein the population databases include information associated with variants in a population, wherein the annotation information includes missense status and germline alteration status associated with a given variant, thereby generating a plurality of annotated variants;
filtering the plurality of annotated variants, wherein the filtering applies a rule set to the annotated variants to retain the detected variants that are non-synonymous somatic single nucleotide variants (SNVs), the rule set comprises:
(i) removing SNVs corresponding to SNPs in a database of germline alterations; and
(ii) removing SNVs not annotated as missense variants, wherein the filtering produces identified non-synonymous somatic SNVs;
counting the identified non-synonymous somatic SNVs to give a tumor mutation value;
determining a number of bases in the genomic regions targeted by the transcriptomic profile in the tumor sample genome; and
calculating a number of non-synonymous somatic SNVs per megabase by dividing the tumor mutation value by the number of bases in the genomic regions targeted by the transcriptomic profile to produce the mutation load.

13. The system of claim 12, wherein the population databases include one or more of a 1000 genomes database, Ensembl variation databases, COSMIC, Human Gene Mutation Database dbSNP, and an Exome Aggregation Consortium (ExAC) database.

14. The system of claim 12 or 13, wherein the database of germline alterations in the dbSNP database.

15. The method of claim 12, wherein the rule set further comprises removing the SNVs present in HLA and Ig genes and removing the SNVs with fewer than 25 total reads prior to (i).

16. The system of claim 12, wherein the rule set further comprises removing SNPs having a reads ratio inconsistent with somatic mutation following step (ii), wherein the reads ratio equals reference allele reads/total reads.

17. The system of claim 12, wherein the number of bases in the genomic regions targeted by the transcriptomic profile used to divide the tumor mutation value is multiplied by the percentage of bases with a desired sequencing depth.

18. The system of claim 17, wherein the desired sequencing depth is 20×.

19. The system of claim 12, wherein the genomic regions targeted by the transcriptomic profile are exons.

20. The system of claim 12, wherein the detecting variants is configured by variant caller parameters, the variant caller parameters including a minimum allele frequency parameter, a strand bias parameter and a data quality stringency parameter.

21. The system of claim 12, wherein, prior to detecting variants, the method comprises aligning the nucleic acid sequence reads obtained from the transcriptomic profiling to a human reference genome_{;} sorting and indexing; re-aligning to remove alignment errors and reference bias; and removing adjacent SNVs and indels.

22. The system of claim 21, wherein the aligning the nucleic acid sequence reads obtained from the transcriptomic profiling to the human reference genome is performed with a spliced mapper.

23. A non-transitory machine-readable storage medium comprising instructions which, when executed by a processor, cause the processor to perform a method analyzing a tumor sample genome for a mutation load, comprising:
detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germ-line variants;
annotating the plurality of detected variants with annotation information from one or more population databases, wherein the population databases include information associated with variants in a population, wherein the annotation information includes missense status and germline alteration status associated with a given variant, thereby generating a plurality of annotated variants;
filtering the plurality of annotated variants, wherein the filtering applies a rule set to the annotated variants to retain the detected variants that are non-synonymous somatic single nucleotide variants (SNVs), the rule set comprises:
(i) removing SNVs corresponding to SNPs in a database of germline alterations; and
(ii) removing SNVs not annotated as missense variants, wherein the filtering produces identified non-synonymous somatic SNVs;
counting the identified non-synonymous somatic SNVs to give a tumor mutation value;
determining a number of bases in the genomic regions targeted by the transcriptomic profile in the tumor sample genome; and
calculating a number of non-synonymous somatic SNVs per megabase by dividing the tumor mutation value by the number of bases in the genomic regions targeted by the transcriptomic profile to produce the mutation load.

24. A method of identifying an individual having a cancer who may benefit from a cancer therapy, the method comprising determining a tumor mutational burden (TMB) rate using RNA sequencing data obtained from a tumor sample from the individual, wherein a TMB rate from the tumor sample that is at or above a reference TMB rate identifies the individual as one who may benefit from the cancer therapy.

25. A method for selecting a cancer therapy for an individual having a cancer, the method comprising determining a TMB rate using RNA sequencing data from a tumor sample from the individual, wherein a TMB rate from the tumor sample that is at or above a reference TMB rate identifies the individual as one who may benefit from the cancer therapy.

26. The method of claim 24 or 25, wherein the TMB rate determined from the tumor sample is at or above the reference TMB rate, and the method further comprises administering to the individual an effective amount of the cancer therapy.

27. The method of claim 24 or 25, wherein the TMB rate determined from the tumor sample is below the reference TMB rate.

28. A method of treating an individual having a cancer, the method comprising:
(a) determining a TMB rate from a tumor sample obtained from the individual, wherein the TMB rate from the tumor sample is at or above a reference TMB rate, and wherein the TMB rate is calculated from RNA sequencing data; and
(b) administering a cancer therapy to the individual.

29. The method of claim 24, 25 or 28, wherein the reference TMB rate is a pre-assigned TMB rate.

30. The method of claim 24, 25 or 28, wherein the reference TMB rate is between about 2 and about 5 mutations per megabase (mut/Mb).

31. The method of claim 24, 25 or 28, wherein the TMB rate using RNA sequencing data reflects a rate of non-synonymous somatic mutations.

32. The method of claim 31, wherein the rate of non-synonymous somatic mutations represents a rate of candidate neoantigens.

33. The method of claim 31, wherein the non-synonymous somatic mutations comprise mutations that have arisen due to RNA editing.

34. The method of claim 24, 25 or 28, wherein the cancer is a cervical kidney renal papillary cell carcinoma (KIRP); breast invasive carcinoma (BRCA); thyroid cancer (THCA); bladder carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head-neck squamous cell carcinoma (HNSC); uterine corpus endometrial carcinoma (UCEC); glioblastoma muitiforine (GBM); esophageal carcinoma (ESCA); stomach adenocarcinoma (STAD); ovarian cancer (OV): rectum adenocarcinoma (READ) or lung, squamous cell carcinoma (LUSC).

35. The method of claim 33, wherein the cancer is lung adenocarcinoma (LUAD); colon adenocarcinoma (COAD), breast invasive carcinoma (BRCA), uterine corpus endometrial carcinoma (UCEC), rectum adenocarcinoma (READ) or lung squamous cell carcinoma (LUSC).

36. The method of claim 24, 25 or 28, wherein the cancer therapy is selected from surgical intervention, radiotherapy, one or more chemotherapeutic agents, one or more PARP inhibitors, and one or more immunotherapeutic agents.

37. The method of claim 36, wherein the one or more immunotherapeutic agents is an immune checkpoint modulator.

38. The method of claim 37, wherein the immune checkpoint modulator interacts with cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof.

39. The method of claim 37, wherein the immune checkpoint modulator is an antibody agent.

40. The method of claim 39, wherein the antibody agent is or comprises a monoclonal antibody or antigen binding fragment thereof.

41. The method of claim 24, 25 or 28, wherein the determining the TMB rate using RNA sequencing data comprises:
detecting variants in a plurality of nucleic acid sequence reads obtained from transcriptomic profiling of the tumor sample to produce a plurality of detected variants, wherein the nucleic acid sequence reads correspond to genomic regions targeted by the transcriptomic profile of the tumor sample, wherein the detected variants include somatic variants and germline variants;
annotating the plurality of detected variants with annotation information from one or more population databases, wherein the population databases include information associated with variants in a population, wherein the annotation information includes missense status and germline alteration status associated with a given variant, thereby generating a plurality of annotated variants;
filtering the plurality of annotated variants, wherein the filtering applies a rule set to the annotated variants to retain the detected variants that are non-synonymous somatic single nucleotide variants (SNVs), the rule set comprises:
(i) removing SNVs corresponding to SNPs in a database of germline alterations; and
(ii) removing SNVs not annotated as missense variants, wherein the filtering produces identified non-synonymous somatic SNVs;
counting the identified non-synonymous somatic SNVs to give a tumor mutation value;
determining a number of bases in the genomic regions targeted by the transcriptomic profile in the tumor sample genome; and
calculating a number of non-synonymous somatic SNVs per megabase by dividing the tumor mutation value by the number of bases in the genomic regions targeted by the transcriptomic profile to produce the mutation load.

42. The method of claim 41, wherein the population databases include one or more of a 1000 genomes database, Ensembl variation databases, COSMIC, Human Gene Mutation Database dbSNP, and an Exome Aggregation Consortium (ExAC) database.

43. The method of claim 41, wherein the database of germline alterations in the dbSNP database.

44. The method of claim 41, wherein the rule set further comprises removing the SNVs present in HLA and Ig genes and removing the SNVs with fewer than 25 total reads prior to (i).

45. The method of claim 41, wherein the rule set further comprises removing SNPs having a reads ratio inconsistent with somatic mutation following step (ii), wherein the reads ratio equals reference allele reads/total reads.

46. The method of claim 41, wherein the number of bases in the genomic regions targeted by the transcriptomic profile used to divide the tumor mutation value is multiplied by the percentage of bases with a desired sequencing depth.

47. The method of claim 46, wherein the desired sequencing depth is 20×.

48. The method of claim 41, wherein the genomic regions targeted by the transcriptomic profile are exons.

49. The method of claim 41, wherein the detecting variants is configured by variant caller parameters, the variant caller parameters including a minimum allele frequency parameter, a strand bias parameter and a data quality stringency parameter.

50. The method of claim 41, wherein, prior to detecting variants, the method comprises aligning the nucleic acid sequence reads obtained from the transcriptomic profiling to a human reference genome; sorting and indexing; re-aligning to remove alignment errors and reference bias; and removing adjacent SNVs and indels.

51. The method of claim 50, wherein the aligning the nucleic acid sequence reads obtained from the transcriptomic profiling to the human reference genome is performed with a spliced mapper.

52. The method of claim 50, wherein the human reference genome is the GRCh38 human reference genome.

53. The method of claim 51, wherein the human reference genome is the GRCh38 human reference genome.
